(19) United States
(12) Patent Application Publication
(10) Pub. No.: US 2026/0295003 A1
(43) Pub. Date: Oct. 1, 2026

(54) ORAL PHARMACEUTICAL COMPOSITION CONTAINING DUALRECEPTOR AGONIST TARGETING GIP AND GLP-1 AND USES THEREOF

(71) Applicant: BRIGHTGENE BIO-MEDICAL TECHNOLOGY CO., LTD., Suzhou (CN)

(72) Inventors: Liu DOU, Suzhou (CN); Banghao YUAN, Suzhou (CN); Yusheng SUI, Suzhou (CN); Xin FENG, Suzhou (CN)

(21) Appl. No.: 19/636,260

(22) Filed: Apr. 1, 2026

Related U.S. Application Data
(63) Continuation of application No. PCT/CN2025/133148, filed on Nov. 6, 2025.

(30) Foreign Application Priority Data
Nov. 6, 2024 (CN) 2024115777064
Jul. 24, 2025 (CN) 2025110240550

(51) Int. Cl. A61K 38/16; A61K 9/20; A61K 9/28

(52) U.S. Cl. CPC A61K 38/16; A61K 9/2013; A61K 9/2027; A61K 9/2054; A61K 9/2059; A61K 9/28

(57) ABSTRACT

An oral pharmaceutical composition containing a compound represented by Formula (I), and uses thereof are provided. Specifically, the oral pharmaceutical composition comprises a compound represented by Formula (I), a co-agent, a compound containing a nitrogen-containing functional group, a medium-chain fatty acid or a salt thereof, and N-(8-[2-hydroxybenzoyl]-amino)octanoic acid (NAC) or a salt thereof.

### CROSS-REFERENCE TO RELATED APPLICATIONS

This application is a continuation application of International Application No. PCT/CN2025/133148, filed on Nov. 6, 2025, which claims the benefit of Chinese Application No. 2025110240550, filed on Jul. 24, 2025 and Chinese Application No. 2024115777064, filed on Nov. 6, 2024, the contents of each of which are incorporated herein by reference.

### REFERENCE TO A SEQUENCE LISTING

The contents of the electronic sequence listing (SRP250180USsequencelist.xml; Size 3,242 bytes; and Date of Creation: Dec. 30, 2025) is herein incorporated by reference in its entirety.

### TECHNICAL FIELD

The present disclosure relates to the field of biomedicine, and in particular, to an oral pharmaceutical composition containing a dual-receptor agonist targeting GIP and GLP-1, and uses thereof.

### BACKGROUND

Glucose-dependent insulinotropic peptide (GIP) is a 42-amino acid gastrointestinal regulatory peptide that exerts physiological effects in glucose homeostasis by stimulating insulin secretion from pancreatic β cells in the presence of glucose and protecting pancreatic β cells. Glucagon-like peptide-1 (GLP-1) is a 37-amino acid peptide that stimulates insulin secretion, protects pancreatic β cells, and inhibits glucagon secretion, gastric emptying, and food intake, resulting in weight loss. GIP and GLP-1 are secreted by K cells and L cells of the small intestinal endothelium, respectively, and are known as incretins. Incretin receptor signaling plays a key physiologically relevant role in glucose homeostasis.

Studies have found that lowering blood glucose concentration to a certain level can restore sensitivity to GIP, indicating that co-agonism of GLP-1R/GIPR can exert a synergistic blood glucose-lowering effect, and dual-receptor agonists targeting GIP and GLP-1 may produce superior blood glucose-lowering effects and insulin secretion stimulation. Therefore, the development of dual-receptor agonists targeting GIP and GLP-1 has become a research hotspot. Currently, dual-receptor agonists targeting GIP and GLP-1 have been developed, such as a series of dual-receptor agonists targeting GIP and GLP-1 disclosed in the prior art document WO 2022/199629 A1. However, due to the easy degradation of dual-receptor agonists targeting GIP and GLP-1 in the gastrointestinal tract, their absorption and stability are both poor. Even when successfully traversing the gastrointestinal barrier, their excessive molecular weight limits their ability to pass through the intestinal mucosa into the bloodstream. Therefore, achieving sufficient bioavailability through oral administration proves challenging.

It is well known that oral administration has better patient compliance, convenience of use, and lower infection risk compared to injection. Meanwhile, drugs absorbed through the intestines enter the venous system to participate in systemic circulation and metabolism in a relatively mild manner, which helps to reduce the acute side effects of the drugs. Therefore, there is an urgent need in the art to develop oral pharmaceutical formulations of dual-receptor agonists targeting GIP and GLP-1.

### SUMMARY

It is an object of the present disclosure to provide an oral pharmaceutical composition. It is another object of the present disclosure to provide uses of the aforementioned oral pharmaceutical composition.

One aspect of the present disclosure is to provide an oral pharmaceutical composition comprising:
(i) a compound represented by Formula (I) or a pharmaceutically acceptable salt, ester, solvate, optical isomer, tautomer, isotopic label, or prodrug thereof;

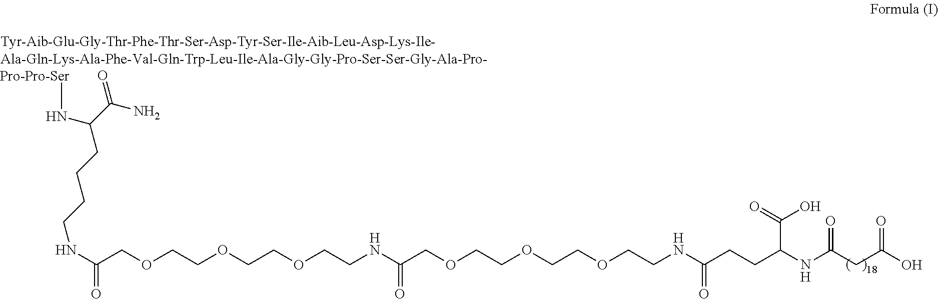

(ii) a co-agent;
(iii) a compound containing a nitrogen-containing functional group;
(iv) a medium-chain fatty acid or a salt thereof; and
(v) N-(8-[2-hydroxybenzoyl]-amino) octanoic acid or a salt thereof;
Wherein the co-agent is a C₆₋₁₀ aromatic ring compound containing a hydrophilic group or a C₅₋₁₀ heteroaromatic ring compound containing a hydrophilic group;
The compound containing a nitrogen-containing functional group contains an amino group, an amine group, or a quaternary ammonium group, and contains at least one substituent group selected from —C(O)OH, —C(O)O⁻, —S(O)₂OH, and —OH.

In one embodiment of the present disclosure, the pharmaceutical composition comprises an active ingredient, wherein the active ingredient is the compound represented by Formula (I).

In one embodiment of the present disclosure, the co-agent or the compound containing a nitrogen-containing functional group is not covalently bonded to the compound represented by Formula (I).

In one embodiment of the present disclosure, the compound containing a nitrogen-containing functional group has a molecular weight of not more than about 500 Da.

In one embodiment of the present disclosure, the pharmaceutical composition comprises not less than about 0.5% w/w of a dual-receptor agonist targeting GIP and GLP-1.

In one embodiment of the present disclosure, the pharmaceutical composition comprises not less than about 0.5% w/w and not more than about 15.0% w/w of the dual-receptor agonist targeting GIP and GLP-1.

In one embodiment of the present disclosure, the pharmaceutical composition comprises not less than about 0.5% w/w and not more than about 10.0% w/w of the dual-receptor agonist targeting GIP and GLP-1.

In one embodiment of the present disclosure, the pharmaceutical composition comprises not less than about 0.5% w/w and not more than about 5.0% w/w of the dual-receptor agonist targeting GIP and GLP-1.

In one embodiment of the present disclosure, the pharmaceutical composition comprises not less than about 20.0% w/w of the medium-chain fatty acid or a salt thereof.

In one embodiment of the present disclosure, the pharmaceutical composition comprises not less than about 25% w/w of the medium-chain fatty acid or a salt thereof.

In one embodiment of the present disclosure, the pharmaceutical composition comprises not more than about 50.0% w/w of the medium-chain fatty acid or a salt thereof.

In one embodiment of the present disclosure, the pharmaceutical composition comprises not more than about 45% w/w of the medium-chain fatty acid or a salt thereof.

In one embodiment of the present disclosure, the pharmaceutical composition comprises not more than about 40% w/w of the medium-chain fatty acid or a salt thereof.

In one embodiment of the present disclosure, the pharmaceutical composition comprises not less than about 20.0% w/w and not more than about 50.0% w/w of the medium-chain fatty acid or a salt thereof.

In one embodiment of the present disclosure, the pharmaceutical composition comprises not less than about 25.0% w/w and not more than about 40% w/w of the medium-chain fatty acid or a salt thereof.

In one embodiment of the present disclosure, the pharmaceutical composition comprises about 20% w/w, 21% w/w, 22% w/w, 23% w/w, 24% w/w, 24.5% w/w, 24.8% w/w, 25% w/w, 25.1% w/w, 25.3% w/w, 25.5% w/w, 26% w/w, 26.5% w/w, 26.8% w/w, 26.9% w/w, 27% w/w, 27.1% w/w, 27.2% w/w, 27.4% w/w, 27.5% w/w, 27.6% w/w, 27.7% w/w, 27.9% w/w, 28% w/w, 28.5% w/w, 28.9% w/w, 29% w/w, 30% w/w, 35% w/w of the medium-chain fatty acid or a salt thereof.

In one embodiment of the present disclosure, the pharmaceutical composition comprises not less than about 20.0% w/w of N-(8-[2-hydroxybenzoyl]-amino) octanoic acid (NAC) or a salt thereof.

In one embodiment of the present disclosure, the pharmaceutical composition comprises not less than about 25% w/w of N-(8-[2-hydroxybenzoyl]-amino) octanoic acid (NAC) or a salt thereof.

In one embodiment of the present disclosure, the pharmaceutical composition comprises not more than about 50.0% w/w of N-(8-[2-hydroxybenzoyl]-amino) octanoic acid (NAC) or a salt thereof.

In one embodiment of the present disclosure, the pharmaceutical composition comprises not more than about 45% w/w of N-(8-[2-hydroxybenzoyl]-amino) octanoic acid (NAC) or a salt thereof.

In one embodiment of the present disclosure, the pharmaceutical composition comprises not more than about 40% w/w of N-(8-[2-hydroxybenzoyl]-amino) octanoic acid (NAC) or a salt thereof.

In one embodiment of the present disclosure, the pharmaceutical composition comprises not less than about 20.0% w/w and not more than about 50% w/w of N-(8-[2-hydroxybenzoyl]-amino) octanoic acid (NAC) or a salt thereof.

In one embodiment of the present disclosure, the pharmaceutical composition comprises not less than about 25.0% w/w and not more than about 40% w/w of N-(8-[2-hydroxybenzoyl]-amino) octanoic acid (NAC) or a salt thereof.

In one embodiment of the present disclosure, the pharmaceutical composition comprises about 20% w/w, 21% w/w, 22% w/w, 23% w/w, 24% w/w, 24.5% w/w, 25% w/w, 25.1% w/w, 25.2% w/w, 25.3% w/w, 25.5% w/w, 26% w/w, 26.5% w/w, 27% w/w, 27.5% w/w, 27.7% w/w, 28% w/w, 28.3% w/w, 28.5% w/w, 29% w/w, 30% w/w, 35% w/w of N-(8-[2-hydroxybenzoyl]-amino) octanoic acid (NAC) or a salt thereof.

In one embodiment of the present disclosure, the pharmaceutical composition comprises not less than about 5.0% w/w of the compound containing a nitrogen-containing functional group.

In one embodiment of the present disclosure, the pharmaceutical composition comprises not less than about 7.0% w/w of the compound containing a nitrogen-containing functional group.

In one embodiment of the present disclosure, the pharmaceutical composition comprises not less than about 8.0% w/w of the compound containing a nitrogen-containing functional group.

In one embodiment of the present disclosure, the pharmaceutical composition comprises not less than about 9.0% w/w of the compound containing a nitrogen-containing functional group.

In one embodiment of the present disclosure, the pharmaceutical composition comprises not more than about 30.0% w/w of the compound containing a nitrogen-containing functional group; for example, not more than about 25% w/w; not more than about 24% w/w; not more than about 23% w/w; not more than about 22% w/w; not more than about 21% w/w; not more than about 20% w/w.

In one embodiment of the present disclosure, the pharmaceutical composition comprises not less than about 5.0% w/w and not more than about 30.0% w/w of the compound containing a nitrogen-containing functional group.

In one embodiment of the present disclosure, the pharmaceutical composition comprises not less than about 9.0% w/w and not more than about 20.0% w/w of the compound containing a nitrogen-containing functional group.

In one embodiment of the present disclosure, the pharmaceutical composition comprises about 5% w/w, 8% w/w, 9% w/w, 10% w/w, 13% w/w, 14% w/w, 15% w/w, 16% w/w, 16.2% w/w, 16.4% w/w, 16.5% w/w, 16.6% w/w, 16.7% w/w, 16.8% w/w, 16.9% w/w, 17% w/w, 17.1% w/w, 17.5% w/w, 17.7% w/w, 17.9% w/w, 18.0% w/w, 18.1% w/w, 18.2% w/w, 18.3% w/w, 18.4% w/w, 18.5% w/w, 18.6% w/w, 18.7% w/w, 18.8% w/w, 18.9% w/w, 19% w/w, 19.5% w/w, 20% w/w, 25% w/w, 30% w/w of the compound containing a nitrogen-containing functional group.

In one embodiment of the present disclosure, the pharmaceutical composition comprises not less than about 5.0% w/w of the co-agent.

In one embodiment of the present disclosure, the pharmaceutical composition comprises not less than about 7.0% w/w of the co-agent.

In one embodiment of the present disclosure, the pharmaceutical composition comprises not less than about 8.0% w/w of the co-agent.

In one embodiment of the present disclosure, the pharmaceutical composition comprises not less than about 9.0% w/w of the co-agent.

In one embodiment of the present disclosure, the pharmaceutical composition comprises not more than about 30.0% w/w of the co-agent.

In one embodiment of the present disclosure, the pharmaceutical composition comprises not more than about 25% w/w of the co-agent.

In one embodiment of the present disclosure, the pharmaceutical composition comprises not more than about 18.0% w/w of the co-agent.

In one embodiment of the present disclosure, the pharmaceutical composition comprises not more than about 16.0% w/w of the co-agent.

In one embodiment of the present disclosure, the pharmaceutical composition comprises not more than about 15.0% w/w of the co-agent.

In one embodiment of the present disclosure, the pharmaceutical composition comprises not less than about 5.0% w/w and not more than about 30.0% w/w of the co-agent.

In one embodiment of the present disclosure, the pharmaceutical composition comprises not less than about 5.0% w/w and not more than about 20.0% w/w of the co-agent.

In one embodiment of the present disclosure, the pharmaceutical composition comprises not less than about 5.0% w/w and not more than about 15.0% w/w of the co-agent.

In one embodiment of the present disclosure, the pharmaceutical composition comprises not less than about 5.0% w/w and not more than about 10.0% w/w of the co-agent.

In one embodiment of the present disclosure, the pharmaceutical composition comprises about 5% w/w, 6% w/w, 7% w/w, 8% w/w, 8.3% w/w, 8.4% w/w, 8.5% w/w, 8.6% w/w, 8.7% w/w, 8.8% w/w, 8.9% w/w, 9% w/w, 9.1% w/w, 9.2% w/w, 9.3% w/w, 9.4% w/w, 9.5% w/w, 9.6% w/w, 9.7% w/w, 9.8% w/w, 9.9% w/w, or 10% w/w of the co-agent.

In one embodiment of the present disclosure, the pharmaceutical composition comprises not less than about 9.0% w/w and not more than about 30.0% w/w of the co-agent.

In one embodiment of the present disclosure, the pharmaceutical composition comprises not less than about 9.0% w/w and not more than about 20.0% w/w of the co-agent.

In one embodiment of the present disclosure, the pharmaceutical composition comprises about 1-50 parts by weight of the compound represented by Formula (I).

In one embodiment of the present disclosure, the pharmaceutical composition comprises about 1-20 parts by weight of the compound represented by Formula (I).

In one embodiment of the present disclosure, the pharmaceutical composition comprises about 1-10 parts by weight of the compound represented by Formula (I).

In one embodiment of the present disclosure, the pharmaceutical composition comprises about 5-10 parts by weight of the compound represented by Formula (I).

In one embodiment of the present disclosure, the pharmaceutical composition comprises about 10-40 parts by weight of the compound represented by Formula (I).

In one embodiment of the present disclosure, the pharmaceutical composition comprises about 10-200 parts by weight of the co-agent.

In one embodiment of the present disclosure, the pharmaceutical composition comprises about 10-100 parts by weight of the co-agent.

In one embodiment of the present disclosure, the pharmaceutical composition comprises about 30-100 parts by weight of the co-agent.

In one embodiment of the present disclosure, the pharmaceutical composition comprises about 50-100 parts by weight of the co-agent.

In one embodiment of the present disclosure, the pharmaceutical composition comprises about 10-500 parts by weight of the compound containing a nitrogen-containing functional group.

In one embodiment of the present disclosure, the pharmaceutical composition comprises about 10-200 parts by weight of the compound containing a nitrogen-containing functional group.

In one embodiment of the present disclosure, the pharmaceutical composition comprises about 30-200 parts by weight of the compound containing a nitrogen-containing functional group.

In one embodiment of the present disclosure, the pharmaceutical composition comprises about 50-150 parts by weight of the compound containing a nitrogen-containing functional group.

In one embodiment of the present disclosure, the pharmaceutical composition comprises about 50-100 parts by weight of the compound containing a nitrogen-containing functional group.

In one embodiment of the present disclosure, the pharmaceutical composition comprises about 100-500 parts by weight of the medium-chain fatty acid or a salt thereof.

In one embodiment of the present disclosure, the pharmaceutical composition comprises about 100-300 parts by weight of the medium-chain fatty acid or a salt thereof.

In one embodiment of the present disclosure, the pharmaceutical composition comprises about 100-200 parts by weight of the medium-chain fatty acid or a salt thereof.

In one embodiment of the present disclosure, the pharmaceutical composition comprises about 150-200 parts by weight of the medium-chain fatty acid or a salt thereof.

In one embodiment of the present disclosure, the pharmaceutical composition comprises about 100-500 parts by weight of N-(8-[2-hydroxybenzoyl]-amino) octanoic acid (NAC) or a salt thereof.

In one embodiment of the present disclosure, the pharmaceutical composition comprises about 100-300 parts by weight of N-(8-[2-hydroxybenzoyl]-amino) octanoic acid (NAC) or a salt thereof.

In one embodiment of the present disclosure, the pharmaceutical composition comprises about 100-200 parts by weight of N-(8-[2-hydroxybenzoyl]-amino) octanoic acid (NAC) or a salt thereof.

In one embodiment of the present disclosure, the pharmaceutical composition comprises about 150-200 parts by weight of N-(8-[2-hydroxybenzoyl]-amino) octanoic acid (NAC) or a salt thereof.

In one embodiment of the present disclosure, each unit dosage form of the pharmaceutical composition comprises about 5-150 mg of the compound represented by Formula (I); for example, 5 mg, 7 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 90 mg, 100 mg, 110 mg, 120 mg, 130 mg, 140 mg, 150 mg.

In one embodiment of the present disclosure, each unit dosage form of the pharmaceutical composition comprises about 5-100 mg of the compound represented by Formula (I); for example, 5 mg, 7 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 90 mg, 100 mg.

In one embodiment of the present disclosure, each unit dosage form of the pharmaceutical composition comprises about 10-200 mg of the co-agent.

In one embodiment of the present disclosure, each unit dosage form of the pharmaceutical composition comprises about 10-100 mg of the co-agent.

In one embodiment of the present disclosure, each unit dosage form of the pharmaceutical composition comprises about 30-100 mg of the co-agent.

In one embodiment of the present disclosure, each unit dosage form of the pharmaceutical composition comprises about 30-70 mg of the co-agent; for example, 30 mg, 40 mg, 55 mg, 50 mg, 60 mg, 65 mg, 70 mg, 75 mg, 80 mg, 90 mg, 100 mg.

In one embodiment of the present disclosure, each unit dosage form of the pharmaceutical composition comprises about 10-500 mg of the compound containing a nitrogen-containing functional group.

In one embodiment of the present disclosure, each unit dosage form of the pharmaceutical composition comprises about 10-200 mg of the compound containing a nitrogen-containing functional group.

In one embodiment of the present disclosure, each unit dosage form of the pharmaceutical composition comprises about 30-150 mg of the compound containing a nitrogen-containing functional group; for example, 30 mg, 40 mg, 50 mg, 60 mg, 70 mg, 100 mg, 120 mg, 150 mg.

In one embodiment of the present disclosure, each unit dosage form of the pharmaceutical composition comprises about 100-500 mg of the medium-chain fatty acid or a salt thereof.

In one embodiment of the present disclosure, each unit dosage form of the pharmaceutical composition comprises about 100-300 mg of the medium-chain fatty acid or a salt thereof.

In one embodiment of the present disclosure, each unit dosage form of the pharmaceutical composition comprises about 130-180 mg of the medium-chain fatty acid or a salt thereof; for example, 100 mg, 110 mg, 130 mg, 150 mg, 160 mg, 180 mg.

In one embodiment of the present disclosure, each unit dosage form of the pharmaceutical composition comprises about 100-500 mg of N-(8-[2-hydroxybenzoyl]-amino) octanoic acid (NAC) or a salt thereof.

In one embodiment of the present disclosure, each unit dosage form of the pharmaceutical composition comprises about 100-300 mg of N-(8-[2-hydroxybenzoyl]-amino) octanoic acid (NAC) or a salt thereof.

In one embodiment of the present disclosure, each unit dosage form of the pharmaceutical composition comprises about 130-180 mg of N-(8-[2-hydroxybenzoyl]-amino) octanoic acid (NAC) or a salt thereof; for example, 100 mg, 110 mg, 130 mg, 150 mg, 160 mg, 180 mg.

In one embodiment of the present disclosure, in the pharmaceutical composition, the mass percentage ratio of the co-agent to the compound containing a nitrogen-containing functional group or a salt thereof is 1:(1-2.5); for example, 1:2, 1:1.

In one embodiment of the present disclosure, in the pharmaceutical composition, the weight ratio of the medium-chain fatty acid or a salt thereof to the N-(8-[2-hydroxybenzoyl]-amino) octanoic acid (NAC) or a salt thereof is 1:(0.8-1.2); for example, 1:1.

In one embodiment of the present disclosure, in the pharmaceutical composition, the weight ratio of the medium-chain fatty acid or a salt thereof, the N-(8-[2-hydroxybenzoyl]-amino) octanoic acid (NAC) or a salt thereof, and the co-agent, to the compound containing a nitrogen-containing functional group is 3:3:1:1, or 3:3:2:2, or 3:3:1:2, or 3:3:2:1.

In one embodiment of the present disclosure, in the pharmaceutical composition, the weight ratio of sodium decanoate, sodium N-(8-[2-hydroxybenzoyl]-amino) caprylate (SNAC), and arginine or lysine, to nicotinamide is 3:3:1:1, or 3:3:2:2, or 3:3:1:2, or 3:3:2:1.

In one embodiment of the present disclosure, in the pharmaceutical composition, the weight ratio of the compound represented by Formula (I), the co-agent, the compound containing a nitrogen-containing functional group, the medium-chain fatty acid or a salt thereof, to the N-(8-[2-hydroxybenzoyl]-amino) octanoic acid (NAC) or a salt thereof is (1-10):(10-100):(10-100):(100-500):(100-500); preferably (1-10):(10-100):(10-100):(100-300):(100-300); preferably (4-10):(30-100):(50-100):(100-200):(100-200); for example, 7:50:50:150:150, or 7:50:100:150:150, or 7:100:50:150:150.

In one embodiment of the present disclosure, in the pharmaceutical composition, the weight ratio of the compound represented by Formula (I), the co-agent, the compound containing a nitrogen-containing functional group, the medium-chain fatty acid or a salt thereof, to the N-(8-[2-hydroxybenzoyl]-amino) octanoic acid (NAC) or a salt thereof is (5-100):(10-100):(10-150):(75-300):(75-300); preferably (5-100):(30-100):(50-100):(100-200):(100-200); for example, 10:50:100:150:150, 20:50:100:150:150, 40:50:100:150:150, 60:50:100:150:150, or 80:50:100:150:150.

In one embodiment of the present disclosure, the compound containing a nitrogen-containing functional group contains an amine group and —C(O)OH, or an amino group and —C(O)OH; or contains an amine group and —S(O)₂OH, or an amino group and —S(O)₂OH; or contains a quaternary ammonium group and —C(O)O⁻; or contains a quaternary ammonium group and —OH; or contains a quaternary ammonium group, —C(O)O⁻, and —OH.

In one embodiment of the present disclosure, the compound containing a nitrogen-containing functional group has a molecular weight ranging from 50 to 250 g/mol, and the molecular weight of the compound containing a nitrogen-containing functional group is calculated in its free form.

In one embodiment of the present disclosure, the compound containing a nitrogen-containing functional group is a C₁₋₈ compound containing an amino group, an amine group, or a quaternary ammonium functional group.

In one embodiment of the present disclosure, the compound containing an amine functional group or a quaternary ammonium functional group is selected from at least one of amino acids, L-carnitine, taurine, betaine, choline, choline chloride, and carnitine. Among them, the amino acids are selected from natural amino acids or synthetic amino acids.

In one embodiment of the present disclosure, the amino acids are selected from at least one of glycine, alanine, valine, leucine, isoleucine, methionine, phenylalanine, tryptophan, proline, serine, threonine, tyrosine, asparagine, glutamine, cysteine, lysine, arginine, histidine, aspartic acid, glutamic acid, sarcosine, γ-aminobutyric acid, ornithine, and citrulline.

In one embodiment of the present disclosure, the compound containing a nitrogen-containing functional group is an amino acid.

In one embodiment of the present disclosure, the compound containing a nitrogen-containing functional group is arginine or lysine; preferably arginine.

In one embodiment of the present disclosure, the pharmaceutical composition contains 1, 2, 3, 4, or 5 kinds of compounds containing a nitrogen-containing functional group.

In one embodiment of the present disclosure, in the co-agent, the hydrophilic group is not a ribosyl group or a deoxyribosyl group.

In one embodiment of the present disclosure, in the co-agent, the C₆-C₁₀ aromatic ring or C₅-C₁₀ heteroaromatic ring is not substituted with a ribosyl group or a deoxyribosyl group.

In one embodiment of the present disclosure, the co-agent is selected from at least one of nicotinamide, adenosine, adenine, guanine, cytosine, trigonelline, and salicylamide.

In one embodiment of the present disclosure, the co-agent is selected from at least one of nicotinamide, adenosine, trigonelline, and salicylamide; preferably nicotinamide.

In one embodiment of the present disclosure, the pharmaceutical composition contains 1, 2, 3, 4, or 5 kinds of co-agents.

In one embodiment of the present disclosure, the salt of N-(8-[2-hydroxybenzoyl]-amino) octanoic acid (NAC) is a sodium salt, potassium salt, or ammonium salt.

In one embodiment of the present disclosure, the salt of N-(8-[2-hydroxybenzoyl]-amino) octanoic acid (NAC) is sodium N-(8-[2-hydroxybenzoyl]-amino) octanoate (SNAC).

In one embodiment of the present disclosure, the medium-chain fatty acid or a salt thereof is a C₆₋₁₄ carboxylic acid or C₆₋₁₄ carboxylate; preferably a C₈₋₁₄ carboxylic acid or C₈₋₁₄ carboxylate salt.

In one embodiment of the present disclosure, the medium-chain fatty acid or a salt thereof is a saturated C₈₋₁₂ straight-chain carboxylic acid or a salt thereof.

In one embodiment of the present disclosure, the medium-chain fatty acid is selected from n-octanoic acid (C₈), n-decanoic acid (C₁₀), n-undecanoic acid (C₁₁), and n-dodecanoic acid (C₁₂); preferably n-decanoic acid (C₁₀).

In one embodiment of the present disclosure, the medium-chain fatty acid salt is a sodium salt, potassium salt, or ammonium salt; preferably a sodium salt.

In one embodiment of the present disclosure, the medium-chain fatty acid salt is sodium n-decanoate (SD).

In one embodiment of the present disclosure, the pharmaceutical composition contains 1, 2, 3, 4, or 5 kinds of medium-chain fatty acids or salts thereof.

In one embodiment of the present disclosure, the pharmaceutical composition comprises:
the compound represented by Formula (I)—about 1-50 parts by weight;
nicotinamide—about 10-200 parts by weight;
arginine and/or lysine—about 10-500 parts by weight;
sodium decanoate—about 100-500 parts by weight; and
sodium N-(8-[2-hydroxybenzoyl]-amino) octanoate (SNAC)—about 100-500 parts by weight.

In one embodiment of the present disclosure, the pharmaceutical composition comprises:
the compound represented by Formula (I)—about 1-50 parts by weight;
nicotinamide—50±10% parts by weight;
arginine and/or lysine—100±10% parts by weight;
sodium decanoate—150±10% parts by weight; and
sodium N-(8-[2-hydroxybenzoyl]-amino) octanoate—150±10% parts by weight.

In one embodiment of the present disclosure, the pharmaceutical composition comprises:
the compound represented by Formula (I)—about 1-50 parts by weight;
nicotinamide—about 50 parts by weight;
arginine and/or lysine—about 100 parts by weight;
sodium decanoate—about 150 parts by weight; and
sodium N-(8-[2-hydroxybenzoyl]-amino) octanoate—about 150 parts by weight.

In one embodiment of the present disclosure, the pharmaceutical composition comprises:
the compound represented by Formula (I)—about 1-50 parts by weight;
nicotinamide—about 10-100 parts by weight;
arginine and/or lysine—about 50-150 parts by weight;
sodium decanoate—about 100-300 parts by weight; and
sodium N-(8-[2-hydroxybenzoyl]-amino) octanoate—about 100-300 parts by weight.

In one embodiment of the present disclosure, the pharmaceutical composition further comprises an excipient in an amount of not more than about 20% w/w.

In one embodiment of the present disclosure, the pharmaceutical composition further comprises not more than about 15% w/w of an excipient.

In one embodiment of the present disclosure, the excipient is selected from one or more of fillers, disintegrants, and lubricants.

In one embodiment of the present disclosure, the excipient is selected from one or more of fillers, disintegrants, lubricants, and binders.

In one embodiment of the present disclosure, the filler is selected from at least one of starch, sugar powder, dextrin, lactose, microcrystalline cellulose, pregelatinized starch, mannitol, and calcium sulfate.

In one embodiment of the present disclosure, the filler is microcrystalline cellulose.

In one embodiment of the present disclosure, the lubricant is selected from at least one of magnesium stearate, colloidal silicon dioxide, talc, polyethylene glycol, and sodium lauryl sulfate.

In one embodiment of the present disclosure, the lubricant is sodium lauryl sulfate.

In one embodiment of the present disclosure, the lubricants are sodium lauryl sulfate and magnesium stearate.

In one embodiment of the present disclosure, the disintegrant is selected from at least one of dry starch, sodium carboxymethyl cellulose, low-substituted carboxymethyl cellulose, crospolyvinylpyrrolidone, croscarmellose sodium, and sodium carboxymethyl starch.

In one embodiment of the present disclosure, the binder is selected from one or more of povidone, crospovidone, hypromellose, and hydroxypropyl cellulose.

In one embodiment of the present disclosure, the binder is povidone, further preferably povidone K90.

In one embodiment of the present disclosure, the disintegrant is sodium carboxymethyl starch.

In one embodiment of the present disclosure, based on 100% w/w of the pharmaceutical composition, the content of the filler is not more than about 15.0% w/w; for example, 14.6% w/w, 14.5% w/w, 14.0% w/w, 13.5% w/w, 13.0% w/w, 12.7% w/w, 12.5% w/w, 12.0% w/w, 11.8% w/w, 11.6% w/w, 10.0% w/w, 9.6% w/w, 9.5% w/w, 9.3% w/w, 9.2% w/w, 9.0% w/w, 8.9% w/w, 8.8% w/w, 8.5% w/w, 8.3% w/w, 7.5% w/w, 7.3% w/w, or 7.0% w/w.

In one embodiment of the present disclosure, based on 100% w/w of the pharmaceutical composition, the content of the lubricant is not more than about 3.0% w/w; for example, 2.7% w/w, 2.4% w/w, 2.5% w/w, 2.2% w/w, 2.0% w/w, 1.9% w/w, 1.7% w/w, 1.6% w/w, 1.4% w/w, 1.3% w/w, 1.2% w/w, 1.1% w/w, or 1.0% w/w.

In one embodiment of the present disclosure, based on 100% w/w of the pharmaceutical composition, the content of the disintegrant is not more than about 5.0% w/w; for example, 5.0% w/w, 4.9% w/w, 4.7% w/w, 4.5% w/w, 4.1% w/w, 4.0% w/w, 3.8% w/w, 3.3% w/w, 3.2% w/w, 3.0% w/w, 2.7% w/w, 2.6% w/w, 2.4% w/w, 2.3% w/w, 2.2% w/w, 2.1% w/w, 2.0% w/w, 1.8% w/w, 1.9% w/w, 1.6% w/w, or 1.5% w/w.

In one embodiment of the present disclosure, based on 100% w/w of the pharmaceutical composition, the content of the binder is not more than about 5.0% w/w; for example, 5.0% w/w, 4.0% w/w, 3.0% w/w, 2.7% w/w, 2.6% w/w, 2.4% w/w, 2.3% w/w, 2.2% w/w, 2.1% w/w, 2.0% w/w, 1.8% w/w, 1.9% w/w, 1.6% w/w, 1.4% w/w, 1.3% w/w, 1.2% w/w, 1.1% w/w, 1.0% w/w, 0.9% w/w, 0.8% w/w, 0.7% w/w, 0.6% w/w, 0.5% w/w, 0.4% w/w, 0.3% w/w, 0.2% w/w, 0.1% w/w.

In one embodiment of the present disclosure, the excipient comprises microcrystalline cellulose, sodium lauryl sulfate, and sodium carboxymethyl starch.

In one embodiment of the present disclosure, based on 100% w/w of the pharmaceutical composition, the excipient comprises microcrystalline cellulose in amount of not less than about 5.0% w/w and not more than about 15.0% w/w.

In one embodiment of the present disclosure, based on 100% w/w of the pharmaceutical composition, the excipient comprises sodium lauryl sulfate in amount of not less than about 0.01% w/w and not more than about 3.0% w/w of.

In one embodiment of the present disclosure, based on 100% w/w of the pharmaceutical composition, the excipient comprises sodium carboxymethyl starch in amount of not less than about 0.1% w/w and not more than about 5.0% w/w.

In one embodiment of the present disclosure, based on 100% w/w of the pharmaceutical composition, the excipient comprises about 5.0-10.0% w/w of microcrystalline cellulose, about 0.01-3.0% w/w of sodium lauryl sulfate, and about 0.1%-5.0% w/w of sodium carboxymethyl starch.

In one embodiment of the present disclosure, the excipient comprises microcrystalline cellulose, sodium lauryl sulfate, sodium carboxymethyl starch, magnesium stearate, and povidone.

In one embodiment of the present disclosure, based on 100% w/w of the pharmaceutical composition, the excipient comprises about 5.0-10.0% w/w of microcrystalline cellulose, about 0.01-3.0% w/w of sodium lauryl sulfate, about 0.1%-5.0% w/w of sodium carboxymethyl starch, about 0.1-3.0% w/w of magnesium stearate, and about 0.1%-5.0% w/w of povidone.

In one embodiment of the present disclosure, the pharmaceutical composition comprises:
the compound represented by Formula (I)—about 1-50 parts by weight;
nicotinamide—about 10-200 parts by weight;
arginine and/or lysine—about 10-500 parts by weight;
sodium decanoate—about 100-500 parts by weight; and
sodium N-(8-[2-hydroxybenzoyl]-amino) octanoate (SNAC)—about 100-500 parts by weight;
excipient—about 12-150 parts by weight.

In one embodiment of the present disclosure, the pharmaceutical composition comprises:
the compound represented by Formula (I)—about 1-50 parts by weight;
nicotinamide—about 10-100 parts by weight;
arginine and/or lysine—about 50-150 parts by weight;
sodium decanoate—about 100-300 parts by weight; and
sodium N-(8-[2-hydroxybenzoyl]-amino) octanoate—about 100-300 parts by weight;
excipient—about 12-150 parts by weight.

In one embodiment of the present disclosure, the pharmaceutical composition comprises:
the compound represented by Formula (I)—about 1-50 parts by weight (e.g., 10, 20, 40 parts by weight);
nicotinamide—50±10% parts by weight;
arginine and/or lysine—100±10% parts by weight;
sodium decanoate—150±10% parts by weight; and
sodium N-(8-[2-hydroxybenzoyl]-amino) octanoate—150±10% parts by weight;
excipient—about 12-150 parts by weight.

In one embodiment of the present disclosure, the pharmaceutical composition comprises:
the compound represented by Formula (I)—about 1-50 parts by weight (e.g., 10, 20, 40 parts by weight);
nicotinamide—about 50 parts by weight;
arginine and/or lysine—about 100 parts by weight;
sodium decanoate—about 150 parts by weight; and
sodium N-(8-[2-hydroxybenzoyl]-amino) octanoate—about 150 parts by weight;
excipient—about 12-150 parts by weight.

In one embodiment of the present disclosure, the pharmaceutical composition comprises:
the compound represented by Formula (I)—about 1-50 parts by weight (e.g., 10, 20, 40 parts by weight);
Nicotinamide—50 parts by weight;
arginine and/or lysine—100 parts by weight;
sodium decanoate—150 parts by weight; and sodium N-(8-[2-hydroxybenzoyl]-amino) octanoate—150 parts by weight; excipient-30-100 parts by weight.

In one embodiment of the present disclosure, the pharmaceutical composition comprises:
the compound represented by Formula (I)—about 1-50 parts by weight (e.g., 10, 20, 40 parts by weight);
nicotinamide—about 50 parts by weight;
arginine and/or lysine—about 100 parts by weight;
sodium decanoate—about 150 parts by weight; and
sodium N-(8-[2-hydroxybenzoyl]-amino) octanoate—about 150 parts by weight;
excipient—about 40-90 parts by weight.

In one embodiment of the present disclosure, the pharmaceutical composition comprises:
the compound represented by Formula (I)—about 5-10 parts by weight;
nicotinamide—about 50-100 parts by weight;
arginine and/or lysine—about 50-100 parts by weight;
sodium decanoate—about 150-200 parts by weight;
sodium N-(8-[2-hydroxybenzoyl]-amino) octanoate—about 150-200 parts by weight; and
excipient—about 30-80 parts by weight.

In one embodiment of the present disclosure, the pharmaceutical composition comprises:
the compound represented by Formula (I)—about 1-50 parts by weight;
nicotinamide—about 10-200 parts by weight;
arginine and/or lysine—about 10-500 parts by weight;
sodium decanoate—about 100-500 parts by weight; and
sodium N-(8-[2-hydroxybenzoyl]-amino) octanoate (SNAC)—about 100-500 parts by weight;
filler—about 10-80 parts by weight;
lubricant—about 1-15 parts by weight; and
disintegrant—about 1-30 parts by weight.

In one embodiment of the present disclosure, the pharmaceutical composition comprises:
the compound represented by Formula (I)—about 5-10 parts by weight;
nicotinamide—about 50-100 parts by weight;
arginine and/or lysine—about 50-100 parts by weight;
sodium decanoate—about 150-200 parts by weight;
sodium N-(8-[2-hydroxybenzoyl]-amino) octanoate—about 150-200 parts by weight;
filler—about 30-60 parts by weight;
lubricant—about 1-10 parts by weight; and
disintegrant—about 5-20 parts by weight.

In one embodiment of the present disclosure, the pharmaceutical composition comprises:
the compound represented by Formula (I)—about 1-50 parts by weight;
nicotinamide—about 10-200 parts by weight;
arginine—about 10-500 parts by weight;
sodium decanoate—about 100-500 parts by weight; and
sodium N-(8-[2-hydroxybenzoyl]-amino) octanoate (SNAC)—about 100-500 parts by weight;
filler—about 10-80 parts by weight;
lubricant—about 1-15 parts by weight; and
disintegrant—about 1-30 parts by weight.

In one embodiment of the present disclosure, the pharmaceutical composition comprises:
the compound represented by Formula (I)—about 5-10 parts by weight;
nicotinamide—about 50-100 parts by weight;
arginine—about 50-100 parts by weight;
sodium decanoate—about 150-200 parts by weight;
sodium N-(8-[2-hydroxybenzoyl]-amino) octanoate—about 150-200 parts by weight;
filler—about 30-60 parts by weight;
lubricant—about 1-10 parts by weight; and
disintegrant—about 5-20 parts by weight.

In one embodiment of the present disclosure, the pharmaceutical composition comprises:
the compound represented by Formula (I)—about 1-50 parts by weight;
nicotinamide—about 10-200 parts by weight;
lysine—about 10-500 parts by weight;
sodium decanoate—about 100-500 parts by weight; and
sodium N-(8-[2-hydroxybenzoyl]-amino) octanoate (SNAC)—about 100-500 parts by weight;
filler—about 10-80 parts by weight;
lubricant—about 1-15 parts by weight; and
disintegrant—about 1-30 parts by weight.

In one embodiment of the present disclosure, the pharmaceutical composition comprises:
the compound represented by Formula (I)—about 5-10 parts by weight;
nicotinamide—about 50-100 parts by weight;
lysine—about 50-100 parts by weight;
sodium decanoate—about 150-200 parts by weight;
sodium N-(8-[2-hydroxybenzoyl]-amino) octanoate (SNAC)—about 150-200 parts by weight;
filler—about 30-60 parts by weight;
lubricant—about 1-10 parts by weight; and
disintegrant—about 5-20 parts by weight.

In one embodiment of the present disclosure, the pharmaceutical composition comprises:
the compound represented by Formula (I)—7±10% parts by weight;
nicotinamide—50±10% parts by weight;
arginine or lysine—50±10% parts by weight;
sodium decanoate—150±10% parts by weight;
sodium N-(8-[2-hydroxybenzoyl]-amino) octanoate (SNAC)—150±10% parts by weight; and
excipient—about 30-80 parts by weight (e.g., 53 parts by weight).

In one embodiment of the present disclosure, the pharmaceutical composition comprises:
the compound represented by Formula (I)—about 7 parts by weight;
nicotinamide—about 50 parts by weight;
arginine or lysine—about 50 parts by weight;
sodium decanoate—about 150 parts by weight;
sodium N-(8-[2-hydroxybenzoyl]-amino) octanoate (SNAC)—about 150 parts by weight; and
excipient—about 30-80 parts by weight (e.g., 53 parts by weight).

In one embodiment of the present disclosure, the pharmaceutical composition comprises:
the compound represented by Formula (I)—about 7 mg;
nicotinamide—about 50 mg;
arginine or lysine—about 50 mg;
sodium decanoate—about 150 mg;
sodium N-(8-[2-hydroxybenzoyl]-amino) octanoate (SNAC)—about 150 mg; and
excipient—about 30-80 mg (e.g., 53 mg).

In one embodiment of the present disclosure, the excipient comprises: filler (preferably microcrystalline cellulose)—39±10% parts by weight; lubricant (preferably sodium lauryl sulfate)—5±10% parts by weight; and disintegrant (preferably sodium carboxymethyl starch)—9±10% parts by weight.

In one embodiment of the present disclosure, the pharmaceutical composition comprises:
the compound represented by Formula (I)—7±10% parts by weight;
nicotinamide—50±10% parts by weight;
arginine—50±10% parts by weight;
sodium decanoate—150±10% parts by weight;
sodium N-(8-[2-hydroxybenzoyl]-amino) octanoate (SNAC)—150±10% parts by weight;
filler—39±10% parts by weight;
lubricant—5±10% parts by weight; and
disintegrant—9±10% parts by weight.

In one embodiment of the present disclosure, the pharmaceutical composition comprises:
the compound represented by Formula (I)—about 7 parts by weight;
nicotinamide—about 50 parts by weight;
arginine—about 50 parts by weight;
sodium decanoate—about 150 parts by weight;
sodium N-(8-[2-hydroxybenzoyl]-amino) octanoate (SNAC)—about 150 parts by weight;
filler—about 39 parts by weight;
lubricant—about 5 parts by weight; and
disintegrant—about 9 parts by weight.

In one embodiment of the present disclosure, the pharmaceutical composition comprises:
the compound represented by Formula (I)—7±10% parts by weight;
nicotinamide—50±10% parts by weight;
lysine—50±10% parts by weight;
sodium decanoate—150±10% parts by weight;
sodium N-(8-[2-hydroxybenzoyl]-amino) octanoate (SNAC)—150±10% parts by weight;
microcrystalline cellulose—39±10% parts by weight;
sodium lauryl sulfate—5±10% parts by weight; and
sodium carboxymethyl starch—9±10% parts by weight.

In one embodiment of the present disclosure, the pharmaceutical composition comprises:
the compound represented by Formula (I)—about 7 parts by weight;
nicotinamide—about 50 parts by weight;
lysine—about 50 parts by weight;
sodium decanoate—about 150 parts by weight;
sodium N-(8-[2-hydroxybenzoyl]-amino) octanoate (SNAC)—about 150 parts by weight;
microcrystalline cellulose—about 39 parts by weight;
sodium lauryl sulfate—about 5 parts by weight; and
sodium carboxymethyl starch—about 9 parts by weight.

In one embodiment of the present disclosure, the pharmaceutical composition comprises:
the compound represented by Formula (I)—7±10% parts by weight;
nicotinamide—50±10% parts by weight;
arginine or lysine—100±10% parts by weight;
sodium decanoate—150±10% parts by weight; and
sodium N-(8-[2-hydroxybenzoyl]-amino) octanoate (SNAC)—150±10% parts by weight.

In one embodiment of the present disclosure, the pharmaceutical composition comprises:
the compound represented by Formula (I)—about 7 parts by weight;
nicotinamide—about 50 parts by weight;
arginine or lysine—about 100 parts by weight;
sodium decanoate—about 150 parts by weight; and
sodium N-(8-[2-hydroxybenzoyl]-amino) octanoate (SNAC)—about 150 parts by weight.

In one embodiment of the present disclosure, the pharmaceutical composition comprises:
about 1.0%-10.0% w/w of the compound represented by Formula (I); and
about 20.0%-40% w/w of sodium N-(8-[2-hydroxybenzoyl]-amino) octanoate (SNAC); and
about 10.0%-20.0% w/w of arginine or lysine; and
about 7.0%-25.0% w/w of nicotinamide; and
about 20.0%-40.0% w/w of sodium decanoate.

In one embodiment of the present disclosure, the pharmaceutical composition comprises:
about 1.0%-2.0% w/w of the compound represented by Formula (I); and
about 20.0%-40% w/w of sodium N-(8-[2-hydroxybenzoyl]-amino) octanoate (SNAC); and
about 10.0%-20.0% w/w of arginine or lysine; and
about 7.0%-25.0% w/w of nicotinamide; and
about 20.0%-40.0% w/w of sodium decanoate.

In one embodiment of the present disclosure, the pharmaceutical composition comprises:
about 1.0%-10.0% w/w of the compound represented by Formula (I); and
about 20.0%-35% w/w of sodium N-(8-[2-hydroxybenzoyl]-amino) octanoate (SNAC); and
about 10.0%-20.0% w/w of arginine or lysine; and
about 7.0%-15.0% w/w of nicotinamide; and
about 20.0%-40.0% w/w of sodium decanoate.

In one embodiment of the present disclosure, the pharmaceutical composition comprises:
about 1.0%-2.0% w/w of the compound represented by Formula (I); and
about 20.0%-35% w/w of sodium N-(8-[2-hydroxybenzoyl]-amino) octanoate (SNAC); and
about 10.0%-15.0% w/w of arginine or lysine; and
about 7.0%-15.0% w/w of nicotinamide; and
about 20.0%-40.0% w/w of sodium decanoate.

In one embodiment of the present disclosure, the pharmaceutical composition comprises:
about 1.0%-2.0% w/w of the compound represented by Formula (I); and
about 20.0%-30% w/w of sodium N-(8-[2-hydroxybenzoyl]-amino) octanoate (SNAC); and
about 10.0%-20.0% w/w of arginine or lysine; and
about 7.0%-10.0% w/w of nicotinamide;
about 20.0%-30.0% w/w of sodium decanoate;
about 5.0-10.0% w/w of microcrystalline cellulose;
about 0.01-3.0% w/w of sodium lauryl sulfate; and
about 0.1%-5.0% w/w of sodium carboxymethyl starch.

In one embodiment of the present disclosure, the pharmaceutical composition comprises:

about 1.37%-1.52% w/w of the compound represented by Formula (I); and
about 29.41%-32.61% w/w of sodium N-(8-[2-hydroxybenzoyl]-amino) octanoate (SNAC); and
about 9.8%-10.87% w/w of arginine or lysine; and
about 9.8%-19.62% w/w of nicotinamide;
about 29.41%-32.61% w/w of sodium decanoate;
about 7.69-8.52% w/w of microcrystalline cellulose;
about 0.9-1% w/w of sodium lauryl sulfate; and
about 1.8%-2% w/w of sodium carboxymethyl starch.

In one embodiment of the present disclosure, the pharmaceutical composition comprises:
the compound represented by Formula (I)—10±10% parts by weight;
nicotinamide—50±10% parts by weight;
L-arginine—100±10% parts by weight;
sodium decanoate—150±10% parts by weight;
sodium N-(8-[2-hydroxybenzoyl]-amino) octanoate (SNAC)—150±10% parts by weight; and
excipient—54±10% parts by weight.

In one embodiment of the present disclosure, the pharmaceutical composition comprises:
the compound represented by Formula (I)—about 10 parts by weight;
nicotinamide—about 50 parts by weight;
L-arginine—about 100 parts by weight;
sodium decanoate—about 150 parts by weight;
sodium N-(8-[2-hydroxybenzoyl]-amino) octanoate (SNAC)—about 150 parts by weight; and
excipient—about 54 parts by weight.

In one embodiment of the present disclosure, the pharmaceutical composition comprises:
the compound represented by Formula (I)—about 10 mg;
nicotinamide—about 50 mg;
L-arginine—about 100 mg;
sodium decanoate—about 150 mg;
sodium N-(8-[2-hydroxybenzoyl]-amino) octanoate (SNAC)—about 150 mg; and
excipient—about 54 mg.

In one embodiment of the present disclosure, the pharmaceutical composition comprises:
the compound represented by Formula (I)—10±10% parts by weight;
nicotinamide—50±10% parts by weight;
L-arginine—100±10% parts by weight;
sodium decanoate—150±10% parts by weight;
sodium N-(8-[2-hydroxybenzoyl]-amino) octanoate (SNAC)—150±10% parts by weight;
microcrystalline cellulose—35.2±10% parts by weight;
sodium lauryl sulfate—4.6±10% parts by weight;
sodium carboxymethyl starch—9.2±10% parts by weight;
povidone—2±10% parts by weight; and
magnesium stearate—3±10% parts by weight.

In one embodiment of the present disclosure, the pharmaceutical composition comprises:
the compound represented by Formula (I)—about 10 parts by weight;
nicotinamide—about 50 parts by weight;
L-arginine—about 100 parts by weight;
sodium decanoate—about 150 parts by weight;
sodium N-(8-[2-hydroxybenzoyl]-amino) octanoate (SNAC)—about 150 parts by weight;
microcrystalline cellulose—about 35.2 parts by weight;
sodium lauryl sulfate—about 4.6 parts by weight;
sodium carboxymethyl starch—about 9.2 parts by weight;
povidone—about 2 parts by weight; and
magnesium stearate—about 3 parts by weight.

In one embodiment of the present disclosure, the pharmaceutical composition comprises:
the compound represented by Formula (I)—20±10% parts by weight;
nicotinamide—50±10% parts by weight;
L-arginine—100±10% parts by weight;
sodium decanoate—150±10% parts by weight;
sodium N-(8-[2-hydroxybenzoyl]-amino) octanoate (SNAC)—150±10% parts by weight; and
excipient—64±10% parts by weight.

In one embodiment of the present disclosure, the pharmaceutical composition comprises:
the compound represented by Formula (I)—about 20 parts by weight;
nicotinamide—about 50 parts by weight;
L-arginine—about 100 parts by weight;
sodium decanoate—about 150 parts by weight;
sodium N-(8-[2-hydroxybenzoyl]-amino) octanoate (SNAC)—about 150 parts by weight; and
excipient—about 64 parts by weight.

In one embodiment of the present disclosure, the pharmaceutical composition comprises:
the compound represented by Formula (I)—20 mg;
nicotinamide—50 mg;
L-arginine—100 mg;
sodium decanoate—150 mg;
sodium N-(8-[2-hydroxybenzoyl]-amino) octanoate (SNAC)—150 mg; and
excipient—64 mg.

In one embodiment of the present disclosure, the pharmaceutical composition comprises:
the compound represented by Formula (I)—20±10% parts by weight;
nicotinamide—50±10% parts by weight;
L-arginine—100±10% parts by weight;
sodium decanoate—150±10% parts by weight;
sodium N-(8-[2-hydroxybenzoyl]-amino) octanoate (SNAC)—150±10% parts by weight;
microcrystalline cellulose—43.2±10% parts by weight;
sodium lauryl sulfate—4.6±10% parts by weight;
sodium carboxymethyl starch—9.2±10% parts by weight;
povidone—4±10% parts by weight; and
magnesium stearate—3±10% parts by weight.

In one embodiment of the present disclosure, the pharmaceutical composition comprises:
the compound represented by Formula (I)—about 20 parts by weight;
nicotinamide—about 50 parts by weight;
L-arginine—about 100 parts by weight;
sodium decanoate—about 150 parts by weight;
sodium N-(8-[2-hydroxybenzoyl]-amino) octanoate (SNAC)—about 150 parts by weight;
microcrystalline cellulose—about 43.2 parts by weight;
sodium lauryl sulfate—about 4.6 parts by weight;
sodium carboxymethyl starch—about 9.2 parts by weight;
povidone—about 4 parts by weight; and
magnesium stearate—about 3 parts by weight.

In one embodiment of the present disclosure, the pharmaceutical composition comprises:
the compound represented by Formula (I)—40±10% parts by weight;
nicotinamide—50±10% parts by weight;
L-arginine—100±10% parts by weight;
sodium decanoate—150 parts by weight;
sodium N-(8-[2-hydroxybenzoyl]-amino) octanoate (SNAC)—150±10% parts by weight; and
excipient—84±10% parts by weight.

In one embodiment of the present disclosure, the pharmaceutical composition comprises:
the compound represented by Formula (I)—about 40 parts by weight;
nicotinamide—about 50 parts by weight;
L-arginine—about 100 parts by weight;
sodium decanoate—about 150 parts by weight;
sodium N-(8-[2-hydroxybenzoyl]-amino) octanoate (SNAC)—about 150 parts by weight; and
excipient—about 84 parts by weight.

In one embodiment of the present disclosure, the pharmaceutical composition comprises:
the compound represented by Formula (I)—about 40 mg;
nicotinamide—about 50 mg;
L-arginine—about 100 mg;
sodium decanoate—about 150 mg;
sodium N-(8-[2-hydroxybenzoyl]-amino) octanoate (SNAC)—about 150 mg; and
excipient—about 84 mg.

In one embodiment of the present disclosure, the pharmaceutical composition comprises:
the compound represented by Formula (I)—40±10% parts by weight;
nicotinamide—50±10% parts by weight;
L-arginine—100±10% parts by weight;
sodium decanoate—150±10% parts by weight;
sodium N-(8-[2-hydroxybenzoyl]-amino) octanoate (SNAC)—150±10% parts by weight;
microcrystalline cellulose—59.2±10% parts by weight;
sodium lauryl sulfate—4.6±10% parts by weight;
sodium carboxymethyl starch—9.2±10% parts by weight;
povidone—8±10% parts by weight; and
magnesium stearate—3±10% parts by weight.

In one embodiment of the present disclosure, the pharmaceutical composition comprises:
the compound represented by Formula (I)—about 40 parts by weight;
nicotinamide—about 50 parts by weight;
L-arginine—about 100 parts by weight;
sodium decanoate—about 150 parts by weight;
sodium N-(8-[2-hydroxybenzoyl]-amino) octanoate (SNAC)—about 150 parts by weight;
microcrystalline cellulose—about 59.2 parts by weight;
sodium lauryl sulfate—about 4.6 parts by weight;
sodium carboxymethyl starch—about 9.2 parts by weight;
povidone—about 8 parts by weight; and
magnesium stearate—about 3 parts by weight.

In one embodiment of the present disclosure, the pharmaceutical composition is a solid dosage form.

In one embodiment of the present disclosure, the pharmaceutical composition is a tablet, capsule, granule, powder, pulvis, drop pill, or pill.

In one embodiment of the present disclosure, the pharmaceutical composition is a tablet, capsule, or granule.

In one embodiment of the present disclosure, the pharmaceutical composition is a tablet.

In one embodiment of the present disclosure, the pharmaceutical composition is a coated tablet.

In one embodiment of the present disclosure, the pharmaceutical composition is the core of a coated tablet.

In one embodiment of the present disclosure, the pharmaceutical composition is a film-coated tablet.

In one embodiment of the present disclosure, the pharmaceutical composition is a coated tablet, and the coating does not contain enteric-coated materials.

In one embodiment of the present disclosure, the pharmaceutical composition is a coated tablet, and the coating is gastric-soluble.

In one embodiment of the present disclosure, each unit dosage form of the pharmaceutical composition comprises 5-150 mg of the compound represented by Formula (I); preferably 5-100 mg. Examples are 5 mg, 7 mg, 10 mg, 15 mg, 20 mg, 25 mg, 30 mg, 35 mg, 40 mg, 50 mg, 55 mg, 60 mg, 65 mg, 70 mg, 75 mg, 80 mg, 90 mg, 100 mg, 110 mg, 120 mg, 130 mg, 140 mg, 150 mg.

In one embodiment of the present disclosure, the strength of the tablet is 1-150 mg, for example, 1 mg, 2 mg, 3 mg, 4 mg, 5 mg, 6 mg, 6.5 mg, 6.8 mg, 7 mg, 7.1 mg, 7.4 mg, 7.5 mg, 7.9 mg, 8 mg, 8.6 mg, 9 mg, 10 mg, 20 mg, 30 mg, 40 mg, 50 mg, 60 mg, 70 mg, 80 mg, 100 mg, 150 mg.

In one embodiment of the present disclosure, the strength of the tablet is about 1-40 mg.

In one embodiment of the present disclosure, the strength of the tablet is about 1-30 mg.

In one embodiment of the present disclosure, the strength of the tablet is about 5-10 mg.

In one embodiment of the present disclosure, the strength of the tablet is 10 mg.

In one embodiment of the present disclosure, the strength of the tablet is 20 mg.

In one embodiment of the present disclosure, the strength of the tablet is 40 mg.

In another aspect, the present disclosure relates to the use of the oral pharmaceutical composition for:
(i) the manufacture of a medicament for treating diseases associated with GIP and/or GLP-1 receptor agonism; and/or
(ii) the treatment of diseases associated with GIP and/or GLP-1 receptor agonism.

In one embodiment of the present disclosure, the diseases associated with GIP and/or GLP-1 receptor agonism are selected from at least one of type 2 diabetes, obesity, hypertension, dyslipidemia, obstructive sleep apnea, non-alcoholic fatty liver disease, and cardiovascular diseases.

In another aspect, the present disclosure relates to a method for treating diseases associated with GIP and/or GLP-1 receptor agonism, comprising administering a therapeutically effective amount of the oral pharmaceutical composition of the present disclosure to a subject in need.

In one embodiment of the present disclosure, the diseases associated with GIP and/or GLP-1 receptor agonism are selected from at least one of type 2 diabetes, obesity, hypertension, dyslipidemia, obstructive sleep apnea, non-alcoholic fatty liver disease, and cardiovascular diseases.

In the oral pharmaceutical composition of the present disclosure, a compound containing an amine group or a quaternary ammonium functional group, a co-agent, a medium-chain fatty acid or a salt thereof, and N-(8-[2-hydroxybenzoyl]-amino) octanoic acid (NAC) or a salt thereof may act together to significantly enhance the oral plasma drug concentration of the composition.

In the oral pharmaceutical composition of the present disclosure, a compound containing a nitrogen-containing functional group and a co-agent act together to significantly enhance the oral plasma drug concentration of the composition, compared to a pharmaceutical composition that does not comprise the compound containing a nitrogen-containing functional group or a co-agent in the formulation.

The oral pharmaceutical composition disclosed in the present disclosure exhibits good stability in gastric fluid.

The tablets prepared from the oral pharmaceutical composition of the present disclosure exhibit high dissolution rate, good stability, and high oral bioavailability.

### BRIEF DESCRIPTION OF THE DRAWINGS

FIG. 1 is a graph showing the absorption of a tablet comprising the compound represented by Formula (I) in beagle dogs following oral administration according to the example of the present disclosure.

FIG. 2 is a graph showing the residual percentage of the formulation powder of the compound represented by Formula (I) over time during treatment with gastric fluid, according to the example of the present disclosure.

FIG. 3 is a graph showing the dissolution results of the compound represented by Formula (I), according to the example of the present disclosure.

### DETAILED DESCRIPTION

### Embodiments

Based on the aforementioned content of the present disclosure, in accordance with the general technical knowledge and conventional means in the art, various other forms of modifications, substitutions, or changes can also be made without departing from the aforementioned basic technical concepts of the present disclosure.

### I. Definitions

Unless otherwise clearly indicated, throughout the strength and claims, the term “comprise” or its variations, such as “comprises” or “comprising”, etc., shall be understood as including the stated elements or components without excluding other elements or other components.

In the present disclosure, a numerical range refers to each integer in the given range. For example, “C₁₋₆” means that the group may have 1 carbon atom, 2 carbon atoms, 3 carbon atoms, 4 carbon atoms, 5 carbon atoms, or 6 carbon atoms; “C₁₋₃” means that the group may have 1 carbon atom, 2 carbon atoms, or 3 carbon atoms. The term “optional” or “optionally” means that the subsequently described event or circumstance may or may not occur, and the description includes the occurrence of the event or circumstance and the non-occurrence of the event or circumstance.

The term “substituted” or “substitution” means that any one or more hydrogen atoms on a specific atom or group are replaced by substituents, provided that the valence state of the specific atom or group is normal and the substituted compound is stable. When the substituent is a keto group (i.e., ═O), it means that two hydrogen atoms are substituted. Unless otherwise specified, the type and number of substituents may be arbitrary on the basis of chemical feasibility.

The term “about” refers to roughly, approximately, near, or around. When the term “about” is used in conjunction with a numerical range, it modifies the range by expanding the boundaries aforementioned and below the stated numerical values. In general, the term “about” is used herein to modify a value with a deviation of 20% aforementioned and below the value.

In the present disclosure, amino acids and their abbreviations and English acronyms are shown in Table 1 below.

TABLE 1

| | Three-letter | One-letter |
|---|---|---|
| Name | abbreviation | abbreviation |
| Alanine | Ala | A |
| Aspartic acid | Asp | D |
| Glutamic acid | Glu | E |
| Phenylalanine | Phe | F |
| Glycine | Gly | G |
| Isoleucine | Ile | I |
| Lysine | Lys | K |
| Leucine | Leu | L |
| Proline | Pro | P |
| Glutamine | Gln | Q |
| Serine | Ser | S |
| Threonine | Thr | T |
| Valine | Val | V |
| Tryptophan | Trp | W |
| Tyrosine | Tyr | Y |
| 2-Aminoisobutyric acid | Aib | / |

### Pharmaceutical Composition

The term “pharmaceutically acceptable” or “medicinally acceptable” refers to those compounds, materials, compositions, and/or dosage forms that are suitable for contact with human and animal tissues within the scope of reasonable medical judgment, without excessive toxicity, irritation, allergic reaction, or other problems or complications, commensurate with a reasonable benefit/risk ratio.

The term “pharmaceutically acceptable salt” refers to a salt that retains the biological effectiveness of the free acid and free base of the specific compound without biological adverse effects. Examples are acid addition salts (including organic acids and inorganic acids) or base addition salts (including organic bases and inorganic bases).

The term “pharmaceutically acceptable ester” refers to an ester that retains the biological effectiveness of the original specific compound without biological adverse effects. The ester group is generally introduced through an organic acid and an alcoholic hydroxyl group. In some embodiments, a peptide is linked to a fatty acid or a derivative thereof via an ester bond to obtain a pharmaceutically acceptable ester.

The term “prodrug”, also referred to as “drug precursor”, refers to a compound that can be converted into an active drug in vivo and exerts its therapeutic effect. Such conversion is affected by the hydrolysis of the prodrug in the blood or its enzymatic conversion to the parent structure in the blood or tissues. In one embodiment of the present disclosure, the prodrug may be an ester.

The terms “polypeptide” and “peptide” are used interchangeably and refer to compounds comprising a series of amino acids linked to each other via amide (or peptide) bonds. Polypeptides typically contain at least 20 amino acid residues. In some embodiments, the polypeptide comprises 20-500 amino acid residues. In some embodiments, one or more modified amino acid residues and/or non-protein amino acid residues.

The term “agonist” refers to a substance (ligand) that activates signaling through the targeted receptor type. Exemplarily, with a GLP-1 receptor as the target receptor, the agonist has activating activity of the GLP-1 receptor, such as a GLP-1 polypeptide or an analog thereof.

The term “dual-receptor agonist targeting GIP and GLP-1” refers to a substance that has activating activity on both GIP and GLP-1 receptors.

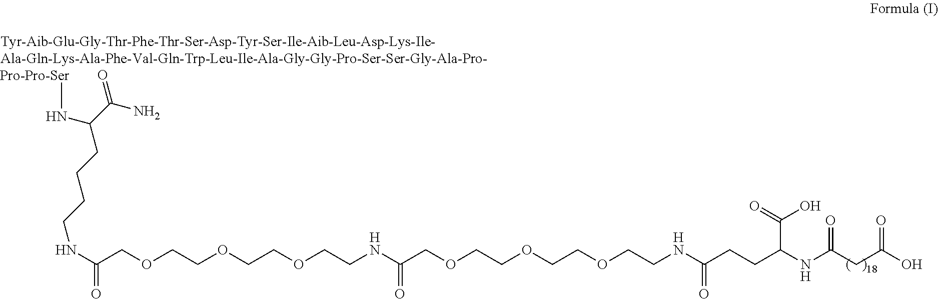

Wherein,

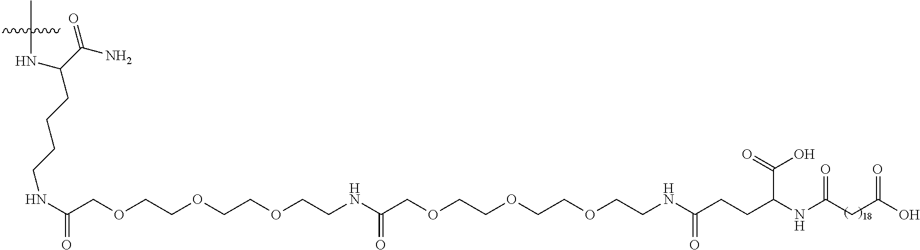

linked to the carboxyl group of the Ser residue at C-terminus.

In the present disclosure, the preparation of the compound represented by Formula (I) can refer to the prior art document WO 2022/199629 A1.

The term “co-agent” refers to a C₆-C₁₀ aromatic ring compound or C₅-C₁₀ heteroaromatic ring compound containing a hydrophilic group in the molecule, wherein the heteroaromatic ring refers to an aromatic ring compound containing at least one heteroatom independently selected from nitrogen, oxygen, and sulfur; the heteroaromatic ring may be a monocyclic or polycyclic system, such as bicyclic system, in which two or more rings exist in the form of a fused ring, bridged ring, or spiro ring, and at least one of which contains one or more heteroatoms. Specific examples of heteroaromatic rings include pyridine, thiophene, imidazole, pyrimidine, furan, pyrazine, thiazole, quinoline, isoquinoline, indole, benzimidazole, imidazopyridine, benzofuran, pyridazine, isoindole, pyridone, but not limited thereto. In some embodiments, the co-agent is selected from at least one of nicotinamide, adenosine, adenine, guanine, cytosine, trigonelline, and salicylamide. In some preferred embodiments, the co-agent is selected from at least one of nicotinamide, adenosine, trigonelline, and salicylamide. In some preferred embodiments, the co-agent is nicotinamide.

The term “hydrophilic group” refers to an atomic group or group in a molecule that can form hydrogen bonds with water molecules, thereby enabling the molecule to be easily soluble in water. These groups are generally polar or ionic and can form hydrogen bonds with the polar oxygen atoms in water molecules, thereby endowing the molecule with hydrophilicity. In the present disclosure, examples of hydrophilic groups include, but are not limited to, hydroxyl, carboxyl, amino, sulfonic acid, phosphate, amide, ribosyl (or deoxyribosyl), carboxylate, etc.

The term “amino” refers to a group composed of one nitrogen atom (N) and two hydrogen atoms (H), which is generally bonded to other groups or molecular structures to form more complex compounds.

The term “amine” is a broad chemical functional group, which refers to a structure with a nitrogen atom that can be linked to one or more hydrocarbon groups (e.g., methyl, ethyl, etc.). The amine group is the basis of amine compounds.

Primary amine: the amine group is linked to one hydrocarbon group (e.g., methylamine: CH₃NH₂).

Secondary amine: the amine group is linked to two hydrocarbon groups (e.g., dimethylamine: CH₃NHCH₃).

Tertiary amine: the amine group is linked to three hydrocarbon groups (e.g., trimethylamine:N(CH₃)₃).

The term “quaternary ammonium group” refers to —N⁺(R)₃, where each R is independently a linear or branched alkyl group, and exemplary quaternary ammonium groups are —N⁺(CH₃)₃ and —N⁺(CH₂CH₃)₃.

In some embodiments, the compound containing a nitrogen-containing functional group is an amino acid, such as glycine, alanine, valine, leucine, isoleucine, methionine, phenylalanine, tryptophan, proline, serine, threonine, tyrosine, asparagine, glutamine, cysteine, lysine, arginine, histidine, aspartic acid, glutamic acid, sarcosine, γ-aminobutyric acid, ornithine, and citrulline. In some preferred embodiments, the compound containing a nitrogen-containing functional group is a basic amino acid. In some preferred embodiments, the compound containing a nitrogen-containing functional group is an amino acid that is positively charged at physiological pH. In some embodiments, the compound containing a nitrogen-containing functional group is a quaternary ammonium cation complex, such as carnitine, L-carnitine, choline, choline chloride, and betaine. In some embodiments, the compound containing a nitrogen-containing functional group is taurine.

In some embodiments, the compound containing a nitrogen-containing functional group is selected from at least one of arginine, lysine, ornithine, citrulline, L-carnitine, taurine, betaine, choline, choline chloride, carnitine, sarcosine, and γ-aminobutyric acid; preferably at least one of arginine, citrulline, lysine, choline, choline chloride, and carnitine.

The term “excipient” refers to a substance added to a pharmaceutical composition to impart a certain shape, size, or physical property, etc., to the drug. Examples of excipients include, but are not limited to, carriers, fillers, binders, disintegrants, lubricants, glidants, flavor masking agents and flavoring agents, stabilizers, dispersants, film-forming agents, preservatives, etc. Suitable excipients can be selected based on factors such as the nature, type, administration route, and therapeutic purpose of the drug. In the present disclosure, the term “excipient” refers to other substances used to impart a certain shape, size, or physical property, etc., to a drug, except for the co-agent, the compound containing an amine group or a quaternary ammonium functional group, the medium-chain fatty acid or a salt thereof, and N-(8-[2-hydroxybenzoyl]-amino) octanoic acid or a salt thereof. In some embodiments, the excipient is a filler, disintegrant, and lubricant. In some embodiments, the excipient is a filler, disintegrant, lubricant, and binder.

The term “filler” refers to an excipient used to increase the weight and volume of tablets and facilitate tableting. Examples of fillers include, but are not limited to, starch, sugar powder, dextrin, lactose, microcrystalline cellulose, pregelatinized starch, mannitol, calcium sulfate, etc. In one embodiment, the filler is microcrystalline cellulose.

The term “disintegrant” refers to an excipient that helps tablets break into fine particles in gastrointestinal fluids. Examples of disintegrants include, but are not limited to, dry starch, sodium carboxymethyl cellulose, low-substituted carboxymethyl cellulose, crospolyvinylpyrrolidone, croscarmellose sodium, and sodium carboxymethyl starch. In one embodiment, the disintegrant is sodium carboxymethyl starch.

The term “lubricant” refers to an excipient used to reduce the friction between particles, prevent raw materials and excipients from adhering to the surface of punches, or reduce the friction between tablets and the wall of die holes. Examples of lubricants include, but are not limited to, magnesium stearate, colloidal silicon dioxide, talc, polyethylene glycol, sodium lauryl sulfate, etc. In one embodiment, the lubricant is sodium lauryl sulfate. In one embodiment, the lubricants are sodium lauryl sulfate and magnesium stearate.

The term “binder” refers to an excipient used to enhance the binding force between powder particles, for example, an excipient that enables loose drug powder to form tablets with certain mechanical strength and integrity during tableting in tablet preparation. Examples of binders include, but are not limited to, starch slurry, pregelatinized starch, hypromellose, hydroxypropyl cellulose, povidone, crospovidone, polyethylene glycol, etc. In one embodiment, the binder is povidone.

Any excipient in the present disclosure may have one or more functions; for example, sodium lauryl sulfate can also be used as a solubilizer in pharmaceutical compositions to improve solubility and promote dissolution.

The term “solid dosage form” refers to a solid pharmaceutical preparation with strong cohesion and shape stability. Forms of solid preparations include, but are not limited to, tablets, capsules, granules, powders, and pulvis. Solid preparations may be conventional preparations, sustained-release or immediate-release preparations (e.g., osmotic pump tablets, sustained-release tablets, sustained-release capsules, fast-disintegrating tablets, etc.). In some embodiments, the pharmaceutical composition of the present disclosure is a tablet, capsule, or granule. In a preferred embodiment, the pharmaceutical composition of the present disclosure is a tablet. The term “tablet” refers to a circular or irregular sheet-like solid preparation made of active pharmaceutical ingredients and suitable excipients. Tablets are mainly oral conventional tablets (including sugar-coated tablets and film-coated tablets), and also include buccal tablets, sublingual tablets, oral patches, chewable tablets, dispersible tablets, soluble tablets, effervescent tablets, vaginal tablets, vaginal effervescent tablets, sustained-release tablets, controlled-release tablets, enteric-coated tablets (including enteric-coated tablets and colon-targeted enteric-coated tablets), and orally disintegrating tablets, etc. The tablets of the present disclosure may be coated by methods well known in the art. In one embodiment, the tablet is a non-enteric-coated tablet, such as a gastric-soluble coated tablet.

The term “parts by weight” refers to the relative weight ratio between ingredients in the formulation.

The term “w/w” refers to the percentage of the mass of an ingredient relative to the total mass of the mixture. In one embodiment of the present disclosure, the pharmaceutical composition is a non-coated tablet, comprising 1.52% w/w of the compound represented by Formula (I), 10.87% w/w of arginine, 10.87% w/w of nicotinamide, 32.61% w/w of SNAC, and 32.61% w/w of sodium decanoate. In one embodiment, the pharmaceutical composition is the core of a coated tablet, comprising 1.52% w/w of the compound represented by Formula (I), 10.87% w/w of arginine, 10.87% w/w of nicotinamide, 32.61% w/w of SNAC, and 32.61% w/w of sodium decanoate. In one embodiment, the pharmaceutical composition is a coated tablet, comprising 3.60% w/w of the compound represented by Formula (I), 18.01% w/w of arginine, 27.01% w/w of SNAC, 27.01% w/w of sodium decanoate, and 9.00% w/w of nicotinamide.

The term “strength” or “drug strength” refers to the amount of a pharmaceutical ingredient contained in a unit dosage form, generally expressed as the weight or content in each unit dosage form, such as each tablet, each capsule, each vial, etc., including indicators such as the content, potency, or loading volume of the main drug. In some embodiments of the present disclosure, the strength of the tablet is 1 mg, 2 mg, 3 mg, 4 mg, 5 mg, 6 mg, 6.5 mg, 6.8 mg, 7 mg, 7.1 mg, 7.4 mg, 7.5 mg, 7.9 mg, 8 mg, 8.6 mg, 9 mg, or 10 mg. In some other embodiments of the present disclosure, the strength of the tablet may also be 15 mg, 20 mg, 25 mg, 30 mg, 35 mg, 40 mg, 45 mg, 50 mg, 55 mg, 60 mg, 65 mg, 70 mg, 75 mg, 80 mg, 90 mg, 100 mg, 110 mg, 120 mg, 130 mg, 140 mg, 150 mg.

The term “unit dosage form” refers to the smallest dosage unit or the smallest packaging unit that can be split in a drug, such as tablets, capsules, pulveres, injections, etc. These units can exist independently physically and can be precisely split in terms of dosage. A unit dosage form generally contains a predetermined amount of active ingredient to produce the desired therapeutic effect and is used in combination with appropriate pharmaceutical excipients.

The term “single-ingredient preparation” refers to a single dosage form that contains only one drug or active ingredient in the formulation.

The term “oral” refers to any form of administration through the mouth, including, but not limited to, oral swallowing (e.g., direct swallowing or swallowing with warm water), oral buccal administration (e.g., placing under the tongue or between the gums and cheeks), brewing for oral administration, and oral chewing.

The term “treatment” includes inhibiting, alleviating, preventing, or eliminating one or more symptoms or side effects associated with the disease, condition, or disorder being treated. The term “effective amount” or “therapeutically effective amount” refers to a dosage sufficient to treat, inhibit, or relieve one or more symptoms of the disease state being treated, or to otherwise provide the desired pharmacological and/or physiological effect. The precise dosage will vary depending on a variety of factors, such as subject-dependent variables (e.g., age, immune system health, etc.), the disease or illness, and the treatment administered. The effect of an effective amount may be relative to a control. These controls are known in the art and discussed herein, and may be, for example, the condition of the subject before or without administration of the drug or drug combination, or in the case of a drug combination, the combined effect may be compared with the effect of administering only one drug.

The term “subject in need” refers to any subject administered with the pharmaceutical composition of the present disclosure; preferably, the subject in need is a mammal, including, but not limited to, domestic animals (e.g., cattle, sheep, cats, dogs, and horses), primates (e.g., humans and non-human primates such as monkeys), rabbits, and rodents (e.g., mice and rats).

The term “therapeutically effective amount” refers to an amount that effectively achieves the desired therapeutic result at the required dose and for the required period of time. The therapeutically effective amount of the compound or pharmaceutical composition of the present disclosure may vary depending upon a variety of factors, such as the disease state, the age, sex, and weight of the individual, and the ability of the immunoadjuvant or pharmaceutical composition to elicit the desired response in the individual.

### II. EXAMPLES

The present disclosure is further illustrated below with reference to examples. The descriptions of specific exemplary embodiments of the present disclosure are for purposes of illustration and exemplification. These descriptions are not intended to limit the present disclosure to the precise forms disclosed; and it will be apparent that many modifications and variations can be made in accordance with the teachings of the specification of the present disclosure. The purpose of selecting and describing the exemplary embodiments is to explain the specific principles of the present disclosure and their practical applications, so that those skilled in the art can implement and utilize different exemplary embodiments of the present disclosure, as well as different selections and modifications.

The experimental methods used in the following examples are conventional, unless otherwise specified.

The dual-receptor agonist targeting GIP and GLP-1 used in the following examples is shown in Table 2 below.

TABLE 2

| Material | Source |
|---|---|
| Compound represented | Self-made by Brightgene Bio-Medical |
| by Formula (I) | Technology Co., Ltd. |

### Example 1: Preparation of Tablets Comprising the Compound Represented by Formula (I)

1) Mixing: Raw materials were taken to prepare the powder of the pharmaceutical preparation system. Each ingredient powder was sieved through a 40-mesh sieve, and then each component was accurately weighed in accordance with the mass ratio below. After weighing, the components were uniformly mixed using a three-dimensional mixer (rotational speed: 50 rpm, time: 300 s).

The formulation of the oral pharmaceutical composition comprising the compound represented by Formula (I) was as follows: 1.52% w/w of the compound represented by Formula (I), 10.87% w/w of arginine, 10.87% w/w of nicotinamide, 32.61% w/w of SNAC, 32.61% w/w of sodium decanoate, 8.52% w/w of microcrystalline cellulose, 1% w/w of sodium lauryl sulfate, and 2% w/w of sodium carboxymethyl starch. Among them, the compound represented by Formula (I): SNAC:sodium decanoate:arginine:nicotinamide=7:150:150:50:50.

2) Tableting: The mixed material was taken for compressing using a flat punch die with a diameter of 10 mm. The feeding amount per tablet was 460 mg. The tablets were removed from the die to obtain 10 mm circular flat tablets containing 7 mg of the compound represented by Formula (I).

### Example 2: Preparation of Tablets Comprising the Compound Represented by Formula (I)

1) Mixing: Raw materials were taken to prepare the powder of the pharmaceutical preparation system. Each ingredient powder was sieved through a 40-mesh sieve, and then each component was accurately weighed in accordance with the mass ratio below. After weighing, the components were uniformly mixed using a three-dimensional mixer (rotational speed: 50 rpm, time: 300 s).

The formulation of the oral pharmaceutical composition containing the compound represented by Formula (I) was as follows: 1.37% w/w of the compound represented by Formula (I), 19.62% w/w of arginine, 9.8% w/w of nicotinamide, 29.41% w/w of SNAC, 29.41% w/w of sodium decanoate, 7.69% w/w of microcrystalline cellulose, 0.9% w/w of sodium lauryl sulfate, and 1.8% w/w of sodium carboxymethyl starch. Among them, the compound represented by Formula (I): SNAC:sodium decanoate:arginine:nicotinamide=7:150:150:100:50.

2) Tableting: The mixed material was taken for compressing using a flat punch die with a diameter of 10 mm. The feeding amount per tablet was 510 mg. The tablets were removed from the die to obtain 10 mm circular flat tablets containing 7 mg of the compound represented by Formula (I).

### Example 3: Preparation of Tablets Comprising the Compound Represented by Formula (I)

1) Mixing: Raw materials were taken to prepare the powder of the pharmaceutical preparation system. Each ingredient powder was sieved through a 40-mesh sieve, and then each component was accurately weighed in accordance with the mass ratio below. After weighing, the components were uniformly mixed using a three-dimensional mixer (rotational speed: 50 rpm, time: 300 s).

The formulation of the pharmaceutical composition containing the compound represented by Formula (I) was as follows: 1.37% w/w of the compound represented by Formula (I), 19.62% w/w of lysine, 9.8% w/w of nicotinamide, 29.41% w/w of SNAC, 29.41% w/w of sodium decanoate, 7.69% w/w of microcrystalline cellulose, 0.9% w/w of sodium lauryl sulfate, and 1.8% w/w of sodium carboxymethyl starch. Among them, the compound represented by Formula (I): SNAC:sodium decanoate:lysine:nicotinamide=7:150:150:100:50.

2) Tableting: The mixed material was taken for compressing using a flat punch die with a diameter of 10 mm. The feeding amount per tablet was 510 mg. The tablets were removed from the die to obtain 10 mm circular flat tablets containing 7 mg of the compound represented by Formula (I).

### Example 4: Preparation of Formulation Powder Comprising the Compound Represented by Formula (I)

10 mg of the lyophilized powder of the compound represented by Formula (I), 214 mg of SNAC, 214 mg of sodium decanoate, 71 mg of arginine, and 71 mg of nicotinamide were accurately weighed separately. The aforementioned powders were mixed in an EP tube to obtain one portion of the formulation powder comprising the compound represented by Formula (I). In the mixed powder, the compound represented by Formula (I): SNAC:sodium decanoate:arginine:nicotinamide=7:150:150:50:50.

### Example 5: Preparation of Tablets Comprising the Compound Represented by Formula (I)

1) Mixing: Raw materials were taken to prepare the powder of the pharmaceutical preparation system. Each ingredient powder was sieved through a 40-mesh sieve, and then each component was accurately weighed in accordance with the mass ratio below. After weighing, the components were uniformly mixed using a three-dimensional mixer (rotational speed: 50 rpm, time: 300 s).

The formulation of the pharmaceutical composition comprising the compound represented by Formula (I) was as follows: 1.37% w/w of the compound represented by Formula (I), 9.80% w/w of arginine, 19.62% w/w of nicotinamide, 29.41% w/w of SNAC, 29.41% w/w of sodium decanoate, 7.69% w/w of microcrystalline cellulose, 0.9% w/w of sodium lauryl sulfate, and 1.8% w/w of sodium carboxymethyl starch. Among them, the compound represented by Formula (I): SNAC:sodium decanoate:arginine:nicotinamide=7:150:150:50:100.

2) Tableting: The mixed material was taken for compressing using a flat punch die with a diameter of 10 mm. The feeding amount per tablet was 510 mg. The tablets were removed from the die to obtain 10 mm circular flat tablets comprising 7 mg of the compound represented by Formula (I).

### Comparative Example 1: Preparation of Tablets Comprising the Compound Represented by Formula (I)

1) Mixing: Raw materials were taken to prepare the powder of the pharmaceutical preparation system. Each ingredient powder was sieved through a 40-mesh sieve, and then each component was accurately weighed in accordance with the mass ratio below. After weighing, the components were uniformly mixed using a three-dimensional mixer (rotational speed: 50 rpm, time: 300 s).

The formulation of the pharmaceutical composition containing the compound represented by Formula (I) was as follows: 1.52% w/w of the compound represented by Formula (I), 65.22% w/w of SNAC, 30.26% w/w of microcrystalline cellulose, 1% w/w of sodium lauryl sulfate, and 2% w/w sodium carboxymethyl starch. Among them, the compound represented by Formula (I): SNAC=7:300.

2) Tableting: The mixed material was taken for compressing using a flat punch die with a diameter of 10 mm. The feeding amount per tablet was 460 mg. The tablets were removed from the die to obtain 10 mm circular flat tablets containing 7 mg of the compound represented by Formula (I).

### Comparative Example 2: Preparation of Tablets Comprising the Compound Represented by Formula (I)

1) Mixing: Raw materials were taken to prepare the powder of the pharmaceutical preparation system. Each ingredient powder was sieved through a 40-mesh sieve, and then each component was accurately weighed in accordance with the mass ratio below. After weighing, the components were uniformly mixed using a three-dimensional mixer (rotational speed: 50 rpm, time: 300 s).

The formulation of the pharmaceutical composition containing the compound represented by Formula (I) was as follows: 1.52% w/w of the compound represented by Formula (I), 10.87% w/w of arginine, 32.61% w/w of SNAC, 32.61% w/w of sodium decanoate, 19.39% w/w of microcrystalline cellulose, 1% w/w of sodium lauryl sulfate, and 2% w/w of sodium carboxymethyl starch. Among them, the compound represented by Formula (I): SNAC:sodium decanoate:arginine=7:150:150:50.

2) Tableting: The mixed material was taken for compressing using a flat punch die with a diameter of 10 mm. The feeding amount per tablet was 460 mg. The tablets were removed from the die to obtain 10 mm circular flat tablets containing 7 mg of the compound represented by Formula (I).

### Comparative Example 3: Preparation of Tablets Comprising the Compound Represented by Formula (I)

1) Mixing: Raw materials were taken to prepare the powder of the pharmaceutical preparation system. Each ingredient powder was sieved through a 40-mesh sieve, and then each component was accurately weighed in accordance with the mass ratio below. After weighing, the components were uniformly mixed using a three-dimensional mixer (rotational speed: 50 rpm, time: 300 s).

The formulation of the pharmaceutical composition containing the compound represented by Formula (I) was as follows: 1.52% w/w of the compound represented by Formula (I), 10.87% w/w of nicotinamide, 32.61% w/w of SNAC, 32.61% w/w of sodium decanoate, 19.39% w/w of microcrystalline cellulose, 1% w/w of sodium lauryl sulfate, and 2% w/w of sodium carboxymethyl starch. Among them, the compound represented by Formula (I): SNAC:sodium decanoate:nicotinamide=7:150:150:50.

2) Tableting: The mixed material was taken for compressing using a flat punch die with a diameter of 10 mm. The feeding amount per tablet was 460 mg. The tablets were removed from the die to obtain 10 mm circular flat tablets containing 7 mg of the compound represented by Formula (I).

### Comparative Example 4: Preparation of Lyophilized Powder Comprising the Compound Represented by Formula (I)

10 mg of the lyophilized powder of the compound represented by Formula (I) was accurately weighed and placed in an EP tube to obtain one portion of the lyophilized powder of the compound represented by Formula (I).

### Comparative Example 5: Preparation of Formulation Powder Comprising the Compound Represented by Formula (I)

10 mg of the lyophilized powder of the compound represented by Formula (I), 214 mg of SNAC, 214 mg of sodium decanoate, and 71 mg of arginine were accurately weighed separately. The aforementioned powders were mixed in an EP tube to obtain one portion of the formulation powder comprising the compound represented by Formula (I). In the mixed powder, the compound represented by Formula (I): SNAC:sodium decanoate:arginine=7:150:150:50.

### Test Example 1

The absorption of the tablets comprising the compound represented by Formula (I) prepared as described above was tested in beagles after oral administration. The specific methods are as follows: Before administration, 1 mL of blank blood was collected using an EDTA-K2 blood collection tube as the 0-hour blood sample for the experiment. The tablets prepared in the Comparative Examples or Examples were administered to beagles via oral gavage, with 3 beagles per group and one tablet per beagle. Subsequently, blood was collected from the forearm veins of beagles using EDTA-K2 blood collection tubes at the following time points after administration: 0.5 h, 1 h, 2 h, and 3 h, with a blood collection volume of 1 mL at each time point. The whole blood collected from beagles was centrifuged. After centrifugation, the supernatant plasma samples were collected and stored at −20° C. for subsequent mass spectrometry detection and analysis.

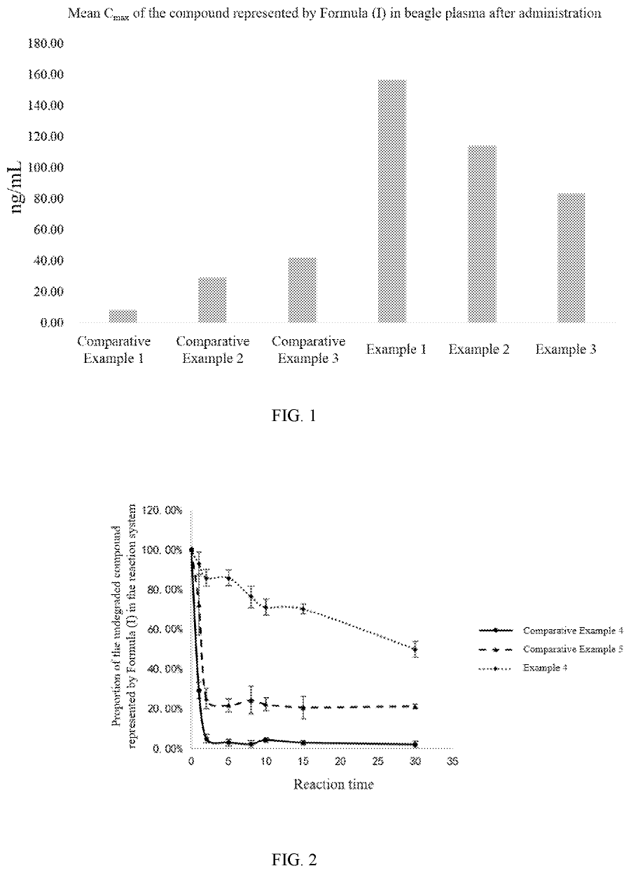

The mass spectrometry detection method was as follows: 20 μL of the plasma sample to be tested or the plasma sample containing the standard of the compound represented by Formula (I) was taken, and 200 μL of a protein precipitant containing glibenclamide as an internal standard was added. The samples were vortexed and centrifuged. Then, 100 μL of the supernatant was transferred to a 96-well plate for injection and analysis. The sample injection volume was 5 μL. The liquid chromatographic column used was ACQUITY UPLC Peptide BEH C18 Column (2.1 mm×50 mm, 1.7 μm). Mobile phase A was 5% acetonitrile containing 0.1% formic acid, and mobile phase B was 95% acetonitrile containing 0.1% formic acid. The flow rate was set at 0.6 mL/min, and the liquid phase gradient was set as follows (time/mobile phase B ratio): 0 min/30%, 1.4 min/75%, 1.6 min/95%, 1.61 min/30%, 1.8 min/30%. The mass spectrometer used was Waters Xevo TQ-S micro. The ionization mode was selected as ESI+, and the scanning mode was MRM. The monitored ion pair for the compound represented by Formula (I) was 1258.3/1837.9, and the monitored ion pair for the internal standard glibenclamide was 494.2/169.1. The results of the oral absorption test of the tablets comprising the compound represented by Formula (I) in beagles are shown in Table 3 and FIG. 1 below. The results showed that the absorption of the compound represented by Formula (I) in beagles in Examples 1-3 was superior to that in the Comparative Examples.

TABLE 3

| | Beagle Cₘₐₓ (ng/mL) | | | |
|---|---|---|---|---|
| Group | 1 | 2 | 3 | Mean |
| Comparative Example 1 | 14.4 | 10.0 | 1.2 | 8.55 |
| Comparative Example 2 | 35.1 | 30.7 | 21.5 | 29.10 |
| Comparative Example 3 | 51.9 | 46.8 | 27.7 | 42.11 |
| Example 1 | 338.2 | 74.3 | 57.2 | 156.57 |
| Example 2 | 145.9 | 128.3 | 68.9 | 114.35 |
| Example 3 | 107.1 | 94.9 | 49.2 | 83.73 |

### Test Example 2

Adequate termination solution (methanol:acetonitrile=1:1), artificial gastric fluid (prepared in accordance with the Chinese Pharmacopoeia), and artificial gastric fluid (without pepsin) were prepared before the test. 1 mL of artificial gastric fluid (without pepsin) was added to each portion of the powders prepared in Comparative Example 4, Comparative Example 5, and Example 5, and vortexed to obtain a stock solution of the compound represented by Formula (I) at a concentration of 10 mg/mL. A certain amount of the stock solution was added to artificial gastric fluid at a ratio of 1:9 and vortexed to obtain a reaction system containing the compound represented by Formula (I) at a concentration of 1 mg/mL, with 3 portions prepared for each group. The time was recorded, and at the time points of 1 min, 2 min, 5 min, 8 min, 10 min, 15 min, 30 min, 45 min, and 60 min, a certain amount of the reaction system was taken, added to the termination solution at a ratio of 1:3, and vortexed. After the samples were centrifuged (10 min, 4° C., 10000 rpm), HPLC was used to detect the content of the compound represented by Formula (I).

20 mg of the compound represented by Formula (I) was accurately weighed, dissolved in 50% acetonitrile, sonicated for 5 min, and then diluted to 20 mL with 50% acetonitrile to obtain the control stock solution. 0.15 mL of the control stock solution was measured and diluted to 10 mL with the pH 7.5 sodium salt buffer to obtain the control sample. For HPLC detection, the chromatographic column used was Hungpu XBTC18 (5 μm, 4.6*150 mm), mobile phase A was 5% acetonitrile-0.1% TFA, and mobile phase B was 95% acetonitrile-0.1% TFA. The HPLC parameters were set as follows: detection wavelength: 220 nm; column temperature: 50° C.; flow rate: 1.0 mL/min; injection volume: 10 μL; isocratic elution for 12 min, mobile phase A: mobile phase B=55:45.

The results of the stability of the compound represented by Formula (I) in gastric fluid in this test example are shown in Table 4 and FIG. 2. The results showed that the excipient combination of SNAC, sodium decanoate, arginine, and nicotinamide can further enhance the stability of the compound represented by Formula (I) in gastric fluid compared to the excipient combination of SNAC, sodium decanoate, and arginine.

TABLE 4
Proportion of the undegraded compound represented
by Formula (I) in the reaction system

| Time (min) | 0 | 1 | 2 | 5 | 8 | 10 | 15 | 30 |
|---|---|---|---|---|---|---|---|---|
| Comparative | 100.00% | 29.29% | 5.02% | 3.02% | 2.07% | 4.29% | 2.92% | 1.91% |
| Example 4 | | | | | | | | |
| Comparative | 100.00% | 72.33% | 25.07% | 21.70% | 24.42% | 22.29% | 20.59% | 21.21% |
| Example 5 | | | | | | | | |
| Example 4 | 100.00% | 92.70% | 85.74% | 85.82% | 76.26% | 71.03% | 70.12% | 49.90% |

### Test Example 3

Adequate dissolution medium (phosphate buffer, pH=6.8) was prepared before the test. The tablets prepared in Comparative Example 2 and Example 1 were taken for in vitro dissolution test. The dissolution was performed with a dissolution medium volume of 500 mL, a dissolution rotation speed set at 50 RPM, and a temperature set at 37° C. (±0.5° C.). The time was recorded, and 1 mL of the sample was taken at the time points of 5 min, 10 min, 15 min, 20 min, 30 min, 45 min, and 60 min, with 1 mL of dissolution medium supplemented after sampling. After the samples were centrifuged, the supernatant was taken for HPLC analysis.

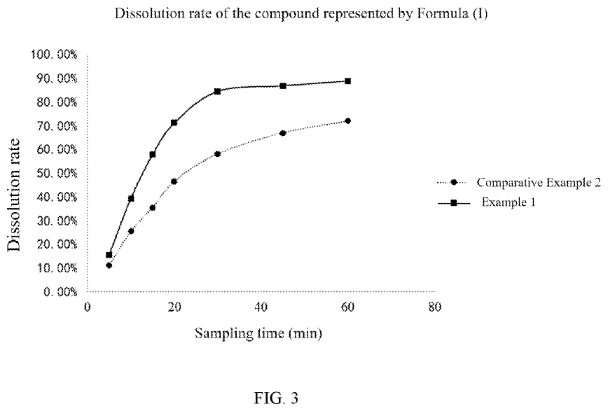

20 mg of BGM0504 was accurately weighed, dissolved in 50% acetonitrile, sonicated for 5 min, and then diluted to 20 mL with 50% acetonitrile to obtain the control stock solution. 0.15 mL of the control stock solution was measured and diluted to 10 mL with the pH 7.5 sodium salt buffer to obtain the control sample. For HPLC detection, the chromatographic column used was Hungpu XBTC18 (5 μm, 4.6*150 mm), mobile phase A was 5% acetonitrile-0.1% TFA, and mobile phase B was 95% acetonitrile-0.1% TFA. The HPLC parameters were set as follows: detection wavelength: 220 nm; column temperature: 50° C.; flow rate: 1.0 mL/min; injection volume: 10 μL; isocratic elution for 12 min, mobile phase A: mobile phase B=55:45. The dissolution test results of this test example are shown in Table 5 and FIG. 3. The results showed that the dissolution of the formulation in Example 1 was significantly superior to that in Comparative Example 2.

TABLE 5
Dissolution rate of the compound represented by Formula (I)

| Sampling time (min) | 5 | 10 | 15 | 20 | 30 | 45 | 60 |
|---|---|---|---|---|---|---|---|
| Comparative | 11.11% | 25.43% | 35.37% | 46.52% | 57.96% | 66.80% | 71.81% |
| Example 2 | | | | | | | |
| Example 1 | 15.58% | 39.31% | 57.77% | 71.13% | 84.41% | 86.74% | 88.71% |

### Example 6: Preparation of Tablets Comprising the Compound Represented by Formula (I)

### 1.1 Formulation

Tablets comprising the compound represented by Formula (I) were prepared in accordance with the formulation in Table 6 below:

TABLE 6

| Component | Content/mg |
|---|---|
| Compound represented by Formula (I) | 20 |
| SNAC | 150 |
| Sodium decanoate | 150 |
| Sodium lauryl sulfate | 4.6 |
| L-arginine | 100 |
| Nicotinamide | 50 |
| Microcrystalline cellulose | 43.2 |
| Povidone K90 | 4 |
| Sodium carboxymethyl starch | 9.2 |
| Magnesium stearate | 3 |
| Total | 534 |

### 1.2 Process

After mixing, the material was compressed into tablets using a direct powder compression process to obtain tablets comprising 20 mg of the compound represented by Formula (I).

### Example 7: Preparation of Tablets Comprising the Compound Represented by Formula (I)

### 7.1 Formulation

Tablets comprising the compound represented by Formula (I) were prepared in accordance with the formulation in Table 7 below:

TABLE 7

| | | |
|---|---|---|
| | Unit dose | Proportion |
| Component | mg/tablet | (%) |

Drug-containing granules

| | | |
|---|---|---|
| Compound represented by Formula (I) | 20 | 3.60 |
| Microcrystalline cellulose | 16 | 2.88 |
| Povidone K90 | 4 | 0.72 |
| Total | 40 | 7.20 |

Blank granules

| | | |
|---|---|---|
| L-arginine | 100 | 18.01 |
| Sodium 8-(2-hydroxybenzamido)caprylate | 150 | 27.01 |
| Sodium decanoate | 150 | 27.01 |
| Microcrystalline cellulose | 27.2 | 4.90 |
| Nicotinamide | 50 | 9.00 |
| Sodium lauryl sulfate | 4.6 | 0.83 |
| Sodium carboxymethyl starch | 9.2 | 1.66 |
| Total | 491 | 88.41 |

Extragranular

| | | |
|---|---|---|
| Magnesium stearate | 3 | 0.54 |
| Core total | 534 | 96.15 |

Coating

| | | |
|---|---|---|
| Film coating premix | 21.36 | 3.85 |
| (gastric-soluble type) | | |
| Purified water | 121.04 | 21.79 |
| Coated tablet total | 555.36 | 100.00 |

### 7.2 Process

The weighed blank granule excipients and drug-containing granule excipients were sieved separately and premixed to obtain blank granule premix powder and drug-containing granule premix powder.

The blank granule premix powder and drug-containing granule premix powder were added to the granulator machine separately for granulation, yielding drug-containing granules and blank granules.

The feeding amounts of the blank granules and magnesium stearate were calculated based on the drug-containing granules, and the materials were weighed. The drug-containing granules and weighed blank granules were mixed, and magnesium stearate was added for further mixing, followed by tableting to obtain tablets with a strength of 20 mg.

The aforementioned tablets can be further coated to obtain 20 mg coated tablets.

### Example 8: Preparation of Tablets Comprising the Compound Represented by Formula (I)

### 1.1 Formulation

Tablets comprising the compound represented by Formula (I) were prepared in accordance with the formulation in Table 8 below:

TABLE 8

| Component | Content/mg |
|---|---|
| Compound represented by Formula (I) | 20 |
| SNAC | 75 |
| Sodium decanoate | 75 |
| Sodium lauryl sulfate | 3.45 |
| L-arginine | 60 |
| Nicotinamide | 30 |
| Microcrystalline cellulose | 29.6 |
| Povidone K90 | 4 |
| Sodium carboxymethyl starch | 4.6 |
| Magnesium stearate | 1.5 |
| Total | 303.15 |

### 1.2 Process

After mixing, the material was compressed into tablets using a direct powder compression process to obtain tablets comprising 20 mg of the compound represented by Formula (I).

### Example 9: Preparation of Tablets Comprising the Compound Represented by Formula (I)

### 1.1 Formulation

Tablets comprising the compound represented by Formula (I) were prepared in accordance with the formulation in Table 9 below:

TABLE 9

| Component | Content/mg |
|---|---|
| Compound represented by Formula (I) | 40 |
| SNAC | 150 |
| Sodium decanoate | 150 |
| Sodium lauryl sulfate | 6.9 |
| L-arginine | 120 |
| Nicotinamide | 60 |
| Microcrystalline cellulose | 59.2 |
| Povidone K90 | 8 |
| Sodium carboxymethyl starch | 9.2 |
| Magnesium stearate | 3 |
| Total | 606.3 |

### 1.2 Process

After thorough mixing, the material was compressed into tablets using a direct powder compression process to obtain tablets comprising 20 mg of the compound represented by Formula (I).

### Example 10: Preparation Comprising Tablets of the Compound Represented by Formula (I)

### 1.1 Formulation

Tablets comprising the compound represented by Formula (I) were prepared in accordance with the formulation in Table 10 below:

TABLE 10

| | | |
|---|---|---|
| | Unit dose | Proportion |
| Component | mg/tablet | (%) |

Drug-containing granules

| | | |
|---|---|---|
| Compound represented by Formula (I) | 40 | 6.70 |
| Microcrystalline cellulose | 32 | 5.36 |
| Povidone K90 | 8 | 1.34 |
| Total | 80 | 13.40 |

Blank granules

| | | |
|---|---|---|
| L-arginine | 100 | 16.75 |
| Sodium 8-(2-hydroxybenzamido)caprylate | 150 | 25.13 |
| Sodium decanoate | 150 | 25.13 |
| Microcrystalline cellulose | 27.2 | 4.56 |
| Nicotinamide | 50 | 8.38 |
| Sodium lauryl sulfate | 4.6 | 0.77 |
| Sodium carboxymethyl starch | 9.2 | 1.54 |
| Total | 491 | 82.25 |

Extragranular

| | | |
|---|---|---|
| Magnesium stearate | 3 | 0.50 |
| Core total | 574 | 96.15 |

Coating

| | | |
|---|---|---|
| Film coating premix | 22.96 | 4.30 |
| (gastric-soluble type) | | |
| Purified water | 130.11 | 24.34 |
| Coated tablet total | 596.96 | 111.67 |

### 1.2 Process

The weighed blank granule excipients and drug-containing granule excipients were sieved separately and premixed to obtain blank granule premix powder and drug-containing granule premix powder.

The blank granule premix powder and drug-containing granule premix powder were added to the granulator machine separately for granulation, yielding drug-containing granules and blank granules.

The feeding amounts of the blank granules and magnesium stearate were calculated based on the drug-containing granules, and the materials were weighed. The drug-containing granules and weighed blank granules were mixed, and magnesium stearate was added for further mixing, followed by tableting to obtain tablets with a strength of 40 mg.

The aforementioned tablets can be further coated to obtain 40 mg film-coated tablets.

### Example 11: Preparation of Tablets Comprising the Compound Represented by Formula (I)

### 1.1 Formulation

Tablets of the compound represented by Formula (I) were prepared in accordance with the formulation in Table 11 below:

TABLE 11

| | | |
|---|---|---|
| | Unit dose | Proportion |
| Component | mg/tablet | (%) |

Drug-containing granules

| | | |
|---|---|---|
| Compound represented by Formula (I) | 10 | 1.87 |
| Microcrystalline cellulose | 8 | 1.50 |
| Povidone K90 | 2 | 0.37 |
| Total | 20 | 3.74 |

Blank granules

| | | |
|---|---|---|
| L-arginine | 100 | 18.71 |
| Sodium 8-(2-hydroxybenzamido)caprylate | 150 | 28.06 |
| Sodium decanoate | 150 | 28.06 |
| Microcrystalline cellulose | 27.2 | 5.09 |
| Nicotinamide | 50 | 9.35 |
| Sodium lauryl sulfate | 4.6 | 0.86 |
| Sodium carboxymethyl starch | 9.2 | 1.72 |
| Total | 491 | 91.85 |

Extragranular

| | | |
|---|---|---|
| Magnesium stearate | 3 | 0.56 |
| Core total | 514 | 96.15 |

Coating

| | | |
|---|---|---|
| Film coating premix (gastric-soluble type) | 20.56 | 3.85 |
| Purified water | 116.51 | 21.79 |
| Coated tablet total | 534.56 | 100.00 |

### 1.2 Process

The weighed blank granule excipients and drug-containing granule excipients were sieved separately and premixed to obtain blank granule premix powder and drug-containing granule premix powder.

The blank granule premix powder and drug-containing granule premix powder were added to the granulator machine separately for granulation, yielding drug-containing granules and blank granules.

The feeding amounts of the blank granules and magnesium stearate were calculated based on the drug-containing granules, and the materials were weighed. The drug-containing granules and weighed blank granules were mixed, and magnesium stearate was added for further mixing, followed by compressing to obtain tablets with a strength of 10 mg.

The aforementioned tablets can be further coated to obtain 10 mg film-coated tablets.

### Comparative Example 6: Preparation of Tablets Comprising the Compound Represented by Formula (I)

### 1.1 Formulation

Tablets comprising the compound represented by Formula (I) were prepared in accordance with the formulation in Table 12 below:

TABLE 12

| Component | Content/mg |
|---|---|
| Compound represented by Formula (I) | 20 |
| SNAC | 0 |
| Sodium decanoate | 300 |
| Sodium lauryl sulfate | 4.6 |
| L-arginine | 100 |
| Nicotinamide | 50 |
| Microcrystalline cellulose | 43.2 |
| Povidone K90 | 4 |
| Sodium carboxymethyl starch | 9.2 |
| Magnesium stearate | 3 |
| Total | 534 |

### 1.2 Process

After mixing, the material was compressed into tablets using a direct powder compression process to obtain tablets comprising 20 mg of the compound represented by Formula (I).

### Comparative Example 7: Preparation of Tablets Comprising the Compound Represented by Formula (I)

### 1.1 Formulation

Tablets comprising the compound represented by Formula (I) were prepared in accordance with the formulation in Table 13 below:

TABLE 13

| Component | Content/mg |
|---|---|
| Compound represented by Formula (I) | 20 |
| SNAC | 300 |
| Sodium decanoate | 0 |
| Sodium lauryl sulfate | 4.6 |
| L-arginine | 100 |
| Nicotinamide | 50 |
| Microcrystalline cellulose | 43.2 |
| Povidone K90 | 4 |
| Sodium carboxymethyl starch | 9.2 |
| Magnesium stearate | 3 |
| Total | 534 |

### 1.2 Process

After mixing, the material was compressed into tablets using a direct powder compression process to obtain tablets comprising 20 mg of the compound represented by Formula (I).

### Test Example 4

Pharmacokinetics tests were conducted using the tablets prepared in Examples 6-11 and Comparative Examples 6 and 7. The test procedures were as follows:

### 1) Animal Information

See Table 14 below.

TABLE 14

| Strain | Sex | Number | Body weight | Weeks of age |
|---|---|---|---|---|
| Beagle | Male | 24 | 6 to 11 kg | ≥6 months |

### 2) Animal Experimental Design

See Table 15 below.

TABLE 15

| | Administration | | |
|---|---|---|---|
| Group | route | Administered tablets | Dosage |
| 1 | PO | Comparative Example 6 | 20 mg |
| 2 | PO | Example 6 | 20 mg |
| 3 | PO | Comparative Example 7 | 20 mg |
| 4 | PO | Example 7 | 20 mg |
| 5 | PO | Example 8 | 20 mg |
| 6 | PO | Example 9 | 40 mg |
| 7 | PO | Example 10 | 40 mg |

### 4) Sample Collection

\textcircled{1} Blood collection time points: PO: 0 h before administration, 15 min, 30 min, 1 h, 2 h, 4 h, 6 h, 8 h, and 24 h after administration;
\textcircled{2} Blood collection site and volume: Extremity veins, 1 mL/time point;
\textcircled{3} Anticoagulant for blood collection tubes: EDTA-2K;
\textcircled{4} Blood collection: Blood samples were collected at the aforementioned set time points and placed in blood collection tubes. The tubes were temporarily placed on wet ice and centrifuged at 4000 g for 5 min (4° C.) within 30 min to separate plasma. The plasma was aliquoted into labeled EP tubes, and the aliquoted samples were stored in an ultra-low temperature refrigerator at −70±10° C. or on dry ice within 30 min until transportation and handover.

### 5) Test Results

The samples were detected using an AB 6500 liquid chromatography-tandem mass spectrometer. The mean values of the detected Cₘₐₓ were calculated, and the results are shown in Table 16 below.

TABLE 16

| | Cₘₐₓ | T_{1/2} | Tₘₐₓ | AUC₀₋₄ | AUC₀₋ₒ₀ |
|---|---|---|---|---|---|
| Group | (ng/mL) | (h) | (h) | (h*ng/mL) | (h*ng/mL) |
| Example 6 | 159 | 37.3 | 1.33 | 2620 | 7447 |
| Example 7 | 275.67 | 33.93 | 2.00 | 4636.67 | 12090.00 |
| Example 8 | 213.4 | 31.23 | 0.50 | 3284.67 | 7466.67 |
| Example 9 | 432.67 | 38.40 | 1.00 | 6460.00 | 17933.33 |
| Example 10 | 463.9 | 29.80 | 2.25 | 8475.00 | 21940.00 |
| Example 11 | 130.5 | 1.08 | / | / | / |
| Comparative | NA | NA | NA | NA | NA |
| Example 6 | | | | | |
| Comparative | 39.59 | 28.87 | 1.33 | 719.33 | 1741.00 |
| Example 7 | | | | | |

Note:
NA in the table indicates not detected; / indicates that the item was not tested.

The results showed that after administering the tablets of Example 6, Comparative Example 6, and Comparative Example 7, respectively, the tablets of Example 6 had the highest Cₘₐₓ in beagles. It can be seen that under the same specification, the addition of SNAC and sodium decanoate to the formulation is beneficial for the increase of oral blood drug concentration in beagles.

1.-39. (canceled)

40. An oral pharmaceutical composition, comprising:
(i) a compound of Formula (I) or a pharmaceutically acceptable salt, ester, solvate, optical isomer, tautomer, isotopic label, or prodrug thereof; 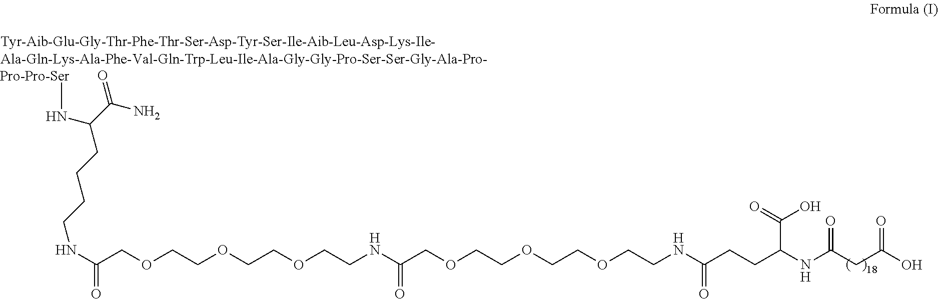
(ii) nicotinamide;
(iii) free amino acid;
(iv) a medium-chain fatty acid or a salt thereof; and
(v) a salt of N-(8-(2-hydroxybenzoyl)amino) caprylic acid (NAC).

41. The composition according to claim 40, wherein the free amino acid is free arginine, free lysine, or a combination thereof.

42. The composition according to claim 40, wherein the medium-chain fatty acid salt is sodium n-decanoate.

43. The composition according to claim 40, wherein the salt of NAC is selected from the group of the sodium, the potassium and the ammonium salt of NAC.

44. The composition according to claim 40, wherein the salt of NAC is SNAC.

45. The composition according to claim 40, wherein the free amino acid, the nicotinamide, or the medium-chain fatty acid or a salt thereof is not covalently bonded to the compound of Formula (I).

46. The composition according to claim 45, wherein the composition comprises:
the compound of Formula (I)—about 1-50 parts by weight;
nicotinamide—about 10-200 parts by weight;
free arginine—about 10-500 parts by weight;
sodium decanoate—about 100-500 parts by weight; and
SNAC—about 100-500 parts by weight.

47. The composition according to claim 45, wherein the composition comprises:
the compound of Formula (I)—about 1-50 parts by weight;
nicotinamide—about 10-100 parts by weight;
free arginine—about 50-150 parts by weight;
sodium decanoate—about 100-300 parts by weight; and
SNAC—about 100-300 parts by weight.

48. The composition according to claim 45, wherein the composition comprises:
the compound of Formula (I)—about 1-50 parts by weight;
nicotinamide—about 50 parts by weight;
free arginine—about 100 parts by weight;
sodium decanoate—about 150 parts by weight; and
SNAC—about 150 parts by weight.

49. The composition according to claim 40, wherein the composition further comprises an excipient with a content of not more than about 20% w/w.

50. The composition according to claim 40, wherein the mass percentage ratio of the nicotinamide to the free amino acid is 1:(1-2.5).

51. The composition according to claim 45, wherein the composition comprises:
the compound of Formula (I)—about 1-50 parts by weight;
nicotinamide—50±10% parts by weight;
free arginine—100±10% parts by weight;
sodium decanoate—150±10% parts by weight; and
SNAC—150±10% parts by weight;
excipient—about 12-150 parts by weight.

52. The composition according to claim 45, wherein the composition comprises:
the compound of Formula (I)—about 10±10% parts by weight;
nicotinamide—50±10% parts by weight;
free L-arginine—100±10% parts by weight;
sodium decanoate—150±10% parts by weight;
SNAC—150±10% parts by weight;
excipient—54±10% parts by weight.

53. The composition according to claim 45, wherein the composition comprises:
the compound of Formula (I)—20=10% parts by weight;
nicotinamide—50±10% parts by weight;
free L-arginine—100±10% parts by weight;
sodium decanoate—150±10% parts by weight;
SNAC—150±10% parts by weight;
excipient—64±10% parts by weight.

54. The composition according to claim 45, wherein the composition comprises:
the compound of Formula (I)—40±10% parts by weight;
nicotinamide—50±10% parts by weight;
free L-arginine—100±10% parts by weight;
sodium decanoate—150±10% parts by weight;
SNAC—150±10% parts by weight;
excipient—84±10% parts by weight.

55. The composition according to claim 40, wherein the composition is a solid dosage form.

56. The composition according to claim 55, wherein the composition is a coated tablet.

57. The composition according to claim 40, wherein each unit dosage form of the composition comprises about 1-150 mg of the compound of Formula (I).

58. A method for treating diseases associated with GIP and/or GLP-1 receptor agonism, comprising the step of: administering a therapeutically effective amount of the composition according to claim 40 to a subject in need.

59. The method according to claim 58, wherein the diseases associated with GIP and/or GLP-1 receptor agonism are selected from type 2 diabetes, obesity, hypertension, dyslipidemia, obstructive sleep apnea, non-alcoholic fatty liver disease, and cardiovascular diseases.


SEQUENCE LISTING (1 sequence)

SEQ ID NO: 1 - protein, 39 aa, synthetic construct
  SITE 2: note = Aib
  SITE 13: note = Aib
  MOTIF 39: note = motified with Fa linked to carboxyl group of Ser at the C-terminal.
YXEGTFTSDYSIXLDKIAQKAFVQWLIAGGPSSGAPPPS